(19) United States
(12) Patent Application Publication
(10) Pub. No.: US 2008/0207695 A1
(43) Pub. Date: Aug. 28, 2008

(54) Thrombin Inhibiting 2-Oxo-1,2,5,6-Tetrahydropyridine Derivatives

(75) Inventors: Ingemar Nilsson, Molndal (SE); Magnus Polla, Molndal (SE)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 11/917,515

(22) PCT Filed: Jun. 14, 2006
(86) PCT No.: PCT/SE2006/000709

§ 371 Date: Dec. 14, 2007

(30) Foreign Application Priority Data
Jun. 17, 2005 (SE) 0501413-9

(51) Int. Cl. A61K 31/444; C07D 213/73; A61K 31/44; A61P 7/02; C07D 401/12

(52) U.S. Cl. 514/335; 546/261; 546/311; 514/352

(57) ABSTRACT

There is provided a compound of formula (I) wherein R¹, R^{2a}, R^{2b}, R^{3a}, R^{3b}, R⁴, R^{5a}, R^{5b}, R⁶ to R⁸, A and G have meanings given in the description, which compounds are useful as, or are useful as prodrugs of, competitive inhibitors of trypsin-like proteases, such as thrombin, and thus, in particular, in the treatment of conditions where inhibition of thrombin is beneficial (e.g. conditions, such as thrombo-embolisms, where inhibition of thrombin is required or desired, and/or conditions where anticoagulant therapy is indicated).

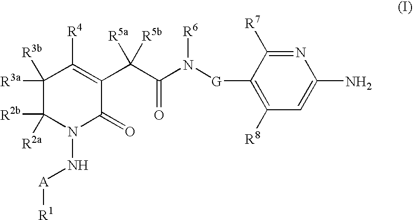

### FIELD OF THE INVENTION

This invention relates to novel pharmaceutically useful compounds, in particular compounds that are, and/or compounds that are metabolised to compounds which are, competitive inhibitors of trypsin-like serine proteases, especially thrombin, their use as medicaments, pharmaceutical compositions containing them and synthetic routes to their production.

### BACKGROUND

Blood coagulation is the key process involved in both haemostasis (i.e. the prevention of blood loss from a damaged vessel) and thrombosis (i.e. the formation of a blood clot in a blood vessel, sometimes leading to vessel obstruction).

Coagulation is the result of a complex series of enzymatic reactions. One of the ultimate steps in this series of reactions is the conversion of the proenzyme prothrombin to the active enzyme thrombin.

Thrombin is known to play a central role in coagulation. It activates platelets, leading to platelet aggregation, converts fibrinogen into fibrin monomers, which polymerise spontaneously into fibrin polymers, and activates factor XIII, which in turn crosslinks the polymers to form insoluble fibrin. Furthermore, thrombin activates factor V, factor VIII and FXI leading to a “positive feedback” generation of thrombin from prothrombin.

By inhibiting the aggregation of platelets and the formation and crosslinking of fibrin, effective inhibitors of thrombin would be expected to exhibit antithrombotic activity. In addition, antithrombotic activity would be expected to be enhanced by effective inhibition of the positive feedback mechanism. Indeed, the convincing antithrombotic effects of a thrombin inhibitor in man has recently been described by S. Schulman et al. in N. Engl. J. Med. 349, 1713-1721 (2003).

### PRIOR ART

The early development of low molecular weight inhibitors of thrombin has been described by Claesson in Blood Coagul. Fibrinol. 5, 411 (1994).

Blombäck et al. (in J. Clin. Lab. Invest. 24, suppl. 107, 59 (1969)) reported thrombin inhibitors based on the amino acid sequence situated around the cleavage site for the fibrinogen Aa chain. Of the amino acid sequences discussed, these authors suggested the tripeptide sequence Phe-Val-Arg (P9-P2-P1, hereinafter referred to as the P3-P2-P1 sequence) would be the most effective inhibitor.

Thrombin inhibitors based on peptidyl derivatives, having cyclic or acyclic basic groups at the PI-position (e.g. groups containing amino, amidino or guanidino functions), are disclosed in, for example, International Patent Application numbers WO 93/11152, WO 93/18060, WO 94/29336, WO 95/23609, WO 95/35309, WO 96/03374, WO 96/25426, WO 96/31504, WO 96/32110, WO 97/02284, WO 97/23499, WO 97/46577, WO 97/49404, WO 98/06740, WO 98/57932, WO 99/29664, WO 00/35869, WO 00/42059, WO 01/87879, WO 02/14270, WO 02/44145 and WO 03/018551, European Patent Application numbers 185 390, 468 231, 526 877, 542 525, 559 046 and 641 779, 648 780, 669 317 and U.S. Pat. No. 4,346,078.

Inhibitors of serine proteases (e.g. thrombin) based on electrophilic ketones in the PI-position are also known, such as the compounds disclosed in European Patent Application numbers 195 212, 362 002, 364 344 and 530 167.

Inhibitors of trypsin-like serine proteases based on C-terminal boronic acid derivatives of arginine (and isothiouronium analogues thereof) are known from European Patent Application number 293 881.

Achiral thrombin inhibitors having, at the P2-position of the molecule, a phenyl group, and a cyclic or acyclic basic group at the P3-position, are disclosed in International Patent Application numbers WO 94/20467, WO 96/06832, WO 96/06849, WO 97/11693, WO 97/24135, WO 98/01422 and WO 01/68605, as well as in Bioorg. Med. Chem. Lett. 7, 1283 (1997).

International Patent Application numbers WO 99/26920 and WO 01/79155 disclose thrombin inhibitors having groups at the P2-position based, respectively, upon 2-aminophenols and 1,4-benzoquinones. Similar, pheno based compounds are also disclosed in International Patent Application numbers WO 01/68605 and WO 02/28825.

Further known inhibitors of thrombin and other trypsin-like serine proteases are based (at the P2-position of the molecule) on the 3-amino-2-pyridone structural unit. For example, compounds based upon 3-amino-2-pyridone, 3-amino-2-pyrazinone, 5-amino-6-pyrimidone, 5-amino-2,6-pyrimidione and 5-amino-1,3,4-triazin-6-one are disclosed in International Patent Application numbers WO 96/18644, WO 97/01338, WO 97/30708, WO 98/16547, WO 99/26926, WO 00/73302, WO 00/75134, WO 01/38323, WO 01/04117, WO 01/70229, WO 01/79262, WO 02/057225, WO 02/064140 and WO 03/29224, U.S. Pat. Nos. 5,668,289 and 5,792,779, as well as in Bioorg. Med. Chem. Lett. 8, 817 (1998) and J. Med. Chem. 41, 4466 (1998).

Thrombin inhibitors based upon the pyridin-2-amine 1-oxide structural unit are disclosed in International Patent Application number WO 02/042272 and in US patent application number US 2003/158218.

Thrombin inhibitors based upon 2-oxo-3-amino-substituted saturated azaheterocycles are disclosed in International Patent Application number WO 95/35313. More recently, thrombin inhibitors have been disclosed that are based upon 4-amino-3-morpholinone (see J. Med. Chem. 46, 1165 (2003)). Further, compounds based upon the structural unit 1-amino-2-pyridone, as well as its di- and tetra-hydrogenated analogues, are described in unpublished international patent application numbers PCT/SE2004/001878 and PCT/SE2005/000124.

None of the above-mentioned documents specifically disclose or suggest compounds based upon 1-amino-2-oxo-1,2,5,6-tetrahydropyridine having, at the PI position, a 2,4-dialkyl-6-aminopyridin-3-yl group in which one or both of the alkyl substituents bears an O-linked substituent.

Moreover, there remains a need for effective inhibitors of trypsin-like serine proteases, such as thrombin. There is also a need for compounds that have a favourable pharmacokinetic profile and/or enhanced oral bioavailability. Such compounds would be expected to be useful as anticoagulants and therefore in the therapeutic treatment of thrombosis and related disorders.

### DISCLOSURE OF THE INVENTION

According to the invention there is provided a compound of formula I

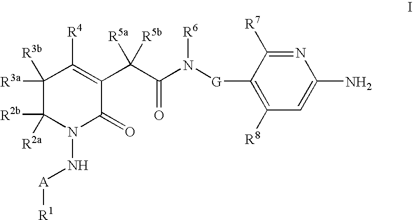

wherein
A represents C(O), S(O), C(O)O (in which latter group the 0 moiety is attached to R¹), C(O)NH, S(O)₂NH (in which latter two groups the NH moiety is attached to R¹), a direct bond or C₁₋₆ alkylene (which latter group is optionally substituted, at the C-atom to which the NH moiety is attached, by C(O)OR^{A} or C(O)N(H)R^{A}); R^{A} represents H or C₁₋₄ alkyl;
R¹ represents
(a) C₁₋₁₀ alkyl, C₂₋₁₀ alkenyl, C₂₋₁₀ alkynyl (which latter three groups are optionally substituted by one or more substituents selected from halo, CN, C₃₋₁₀ cycloalkyl (optionally substituted by one or more substituents selected from halo, OH, ═O, C₁₋₆ alkyl, C₁₋₆ alkoxy and aryl), OR^{9a}, S(O)R^{9b}, S(O)₂N(R^{9c})(R^{9d}), N(R^{9e})S(O)₂R^{9f}, N(R^{9g})(R^{9h}), B¹—C(O)—B²—R⁹ⁱ, aryl and Het¹),
(b) C₃₋₁₀ cycloalkyl or C₄₋₁₀ cycloalkenyl, which latter two groups are optionally substituted by one or more substituents selected from halo, ═O, CN, C₁₋₁₀ alkyl, C₃₋₁₀ cycloalkyl (optionally substituted by one or more substituents selected from halo, OH, ═O, C₁₋₆ alkyl, C₁₋₆ alkoxy and aryl), OR^{9a}, S(O)ₙR^{9b}, S(O)₂N(R^{9c})(R^{9d}), N(R^{9e})S(O)₂R^{9f}, N(R^{9g})(R^{9h}), B³—C(O)—B⁴—R⁹ⁱ, aryl and Het²,
(c) aryl, or
(d) Het³;
   R^{9a} to R⁹ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, aryl and Het⁴),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, aryl and Het⁵),
(d) aryl or
(e) Het⁶,
   provided that R^{9b} does not represent H when n is 1 or 2;
   R^{2a}, R^{2b}, R^{3a} and R^{3b} independently represent H, F, C₁₋₃ alkyl or (CH₂)₀₋₃O(C₁₋₃ alkyl) (which latter two groups are optionally substituted by one OH group or one or more F atoms), or one of R^{2a} and R^{2b}, together with one of R^{3a} and R^{3b}, represents C₁₋₄ n-alkylene;
   R⁴ represents C₁₋₄ alkyl optionally substituted by one or more halo substituents;
   R^{5a} and R^{5b} independently represent H, F or methyl (which latter group is optionally substituted by one or more F atoms);
   R⁶ represents H or C₁₋₄ alkyl (which latter group is optionally substituted by one or more substituents selected from halo and OH),
   G represents C₁₋₄ alkylene;
   R⁷ and R⁸ independently represent C₁₋₄ alkyl optionally substituted by OR¹⁰, provided that at least one of R⁷ and R⁸ is substituted by OR¹⁰;
   R¹⁰ represents H, —C(O)—X—R¹¹ or C₁₋₆ alkyl (which latter group is optionally substituted by one or more substituents selected from halo and C₁₋₃ alkoxy);
   X represents a direct bond, O, S or NH;
   R¹¹ represents
(a) C₁₋₁₀ alkyl, C₂₋₁₀ alkenyl, C₂₋₁₀ alkynyl (which latter three groups are optionally substituted by one or more substituents selected from halo, CN, C₃₋₁₀ cycloalkyl, C₄₋₁₀ cycloalkenyl (which latter two groups are optionally substituted by one or more substituents selected from halo and C₁₋₄ alkyl), OR^{12a}, C(O)OR^{12b}, C(O)N(R^{12c})(R^{12d}), aryl and Het⁷),
(b) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, aryl and Het⁸),
(c) aryl or
(d) Het⁹;
   R^{12a} to R^{12d} independently represent H or C₁₋₆ alkyl; each aryl independently represents a C₆₋₁₀ carbocyclic aromatic group, which group may comprise either one or two rings and may be substituted by one or more substituents selected from
(a) halo,
(b) CN,
(c) C₁₋₁₀ alkyl, C₂₋₁₀ alkenyl, C₂₋₁₀ alkynyl (which latter three groups are optionally substituted by one or more substituents selected from halo, OH, C₃₋₁₀ cycloalkyl, C₄₋₁₀ cycloalkenyl (which latter two groups are optionally substituted by one or more substituents selected from halo and C₁₋₄ alkyl), C₁₋₆ alkoxy, C(O)OH, C(O)O—C₁₋₆ alkyl, C(O)NH₂, phenyl (which latter group is optionally substituted by halo) and Het¹⁰),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het¹⁰),
(e) OR^{13a},
(f) S(O)ₚR^{13b},
(g) S(O)₂N(R^{3c})(R^{13d}),
(h) N(R^{13e})S(O)₂R^{13f},
(i) N(R^{13g})(R^{13h}),
j) B⁵—C(O)—B⁶—R¹³ⁱ,
(k) phenyl (which latter group is optionally substituted by halo),
(l) Het¹² and
(m) Si(R^{14a})(R^{14b})(R^{14c});
   R^{13a} to R¹³ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₃₋₁₀ cycloalkyl, C₄₋₁₀ cycloalkenyl (which latter two groups are optionally substituted by one or more substituents selected from halo and C₁₋₄ alkyl), C₁₋₆ alkoxy, phenyl (which latter group is optionally substituted by halo) and Het¹³),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het¹⁴),
(d) phenyl (which latter group is optionally substituted by halo) or
(e) Het¹⁵,
   provided that R^{13b} does not represent H when p is 1 or 2;
   Het¹ to Het¹⁵ independently represent 4- to 14-membered heterocyclic groups containing one or more heteroatoms selected from oxygen, nitrogen and/or sulfur, which heterocyclic groups may comprise one, two or three rings and may be substituted by one or more substituents selected from
(a) halo,
(b) CN,
(c) C₁₋₁₀ alkyl, C₂₋₁₀ alkenyl, C₂₋₁₀ alkynyl (which latter three groups are optionally substituted by one or more substituents selected from halo, OH, C₃₋₁₀ cycloalkyl, C₄₋₁₀ cycloalkenyl (which latter two groups are optionally substituted by one or more substituents selected from halo and C₁₋₄ alkyl), C₁₋₆ alkoxy, C(O)OH, C(O)O—C₁₋₆ alkyl, C(O)NH₂, phenyl (which latter group is optionally substituted by halo) and Het^{a}),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{b}),
(e) ═O,
(f) OR^{15a},
(g) S(O)_{q}R^{15b},
(h) S(O)₂N(R^{15c})(R^{15d}),
(i) N(R^{15e})S(O)₂R^{15f},
(j) N(R^{15g})(R^{15h}),
(k) B⁷—C(O)—B⁸—R¹⁵ⁱ,
(l) phenyl (which latter group is optionally substituted by halo),
(m) Het^{c} and
(n) Si(R^{16a})(R^{16b})(R^{16c});
   R^{15a} to R¹⁵ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₃₋₁₀ cycloalkyl, C₄₋₁₀ cycloalkenyl (which latter two groups are optionally substituted by one or more substituents selected from halo and C₁₋₄ alkyl), C₁₋₆ alkoxy, phenyl (which latter group is optionally substituted by halo) and Het^{d}),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{e}),
(d) phenyl (which latter group is optionally substituted by halo) or
(e) Het^{f},
   provided that R^{15b} does not represent H when q is 1 or 2;
   Het^{a} to Het^{f} independently represent 5- or 6-membered heterocyclic groups containing one to four heteroatoms selected from oxygen, nitrogen and/or sulfur, which heterocyclic groups may be substituted by one or more substituents selected from halo, ═O and C₁₋₆ alkyl;
   B¹ to B⁸ independently represent a direct bond, O, S, NH or N—C₁₋₄ alkyl; n, p and q independently represent 0, 1 or 2;
   R^{14a}, R^{14b}, R^{14c}, R^{16a}, R^{16b} and R^{16c} independently represent C₁₋₆ alkyl or phenyl (which latter group is optionally substituted by halo or C₁₋₄ alkyl);
   unless otherwise specified
(i) alkyl, alkenyl, alkenyl, cycloalkyl, cycloalkenyl, alkylene and alkenylene groups, as well as the alkyl part of alkoxy groups, may be substituted by one or more halo atoms, and
(ii) cycloalkyl and cycloalkenyl groups may comprise one or two rings and may additionally be ring-fused to one or two phenyl groups;
   or a pharmaceutically-acceptable derivative thereof,
   which compounds are referred to hereinafter as “the compounds of the invention”.

The term “pharmaceutically-acceptable derivatives” includes pharmaceutically-acceptable salts (e.g. acid addition salts).

For the avoidance of doubt, the definitions of the terms aryl, alkyl, alkenyl, alkynyl, cycloalkyl, cycloalkenyl, alkylene, alkenylene and alkoxy groups provided above apply, unless otherwise stated, at each usage of such terms herein.

The term “halo”, when used herein, includes fluoro, chloro, bromo and iodo.

Heterocyclic (Het¹ to Het¹⁵ and Het^{a} to Het^{f}) groups may be fully saturated, partly unsaturated, wholly aromatic or partly aromatic in character. Values of heterocyclic (Het¹ to Het¹⁵ and Het^{a} to Het^{f}) groups that may be mentioned include 1-azabicyclo[2.2.2]octanyl, benzimidazolyl, benzo[c]isoxazolidinyl, benzisoxazolyl, benzodioxanyl, benzodioxepanyl, benzodioxolyl, benzofuranyl, benzofurazanyl, benzomorpholinyl, 2,1,3-benzoxadiazolyl, benzoxazolidinyl, benzoxazolyl, benzopyrazolyl, benzo[e]pyrimidine, 2,1,3-benzothiadiazolyl, benzothiazolyl, benzothienyl, benzotriazolyl, chromanyl, chromenyl, cinnolinyl, 2,3-dihydrobenzimidazolyl, 2,3-dihydrobenzo[b]furanyl, 1,3-dihydrobenzo-[c]furanyl, 1,3-dihydro-2,1-benzisoxazolyl 2,3-dihydropyrrolo[2,3-b]pyridinyl, dioxanyl, furanyl, hexahydropyrimidinyl, hydantoinyl, imidazolyl, imidazo[1,2-a]pyridinyl, imidazo[2,3-b]thiazolyl, indolyl, isoquinolinyl, isoxazolidinyl, isoxazolyl, maleimido, morpholinyl, naphtho[1,2-b]furanyl, oxadiazolyl, 1,2- or 1,3-oxazinanyl, oxazolyl, phthalazinyl, pterazinyl, piperidinyl, purinyl, pyranyl, pyrazinyl, pyrazolyl, pyridinyl, pyrimidinyl, pyrrolidinonyl, pyrrolidinyl, pyrrolinyl, pyrrolo[2,3-b]pyridinyl, pyrrolo[5,1-b]pyridinyl, pyrrolo[2,3-c]pyridinyl, pyrrolyl, quinazolinyl, quinolinyl, sulfolanyl, 3-sulfolenyl, 4,5,6,7-tetrahydrobenzimidazolyl, 4,5,6,7-tetrahydrobenzopyrazolyl, 5,6,7,8-tetrahydro-benzo[e]pyrimidine, tetrahydrofuranyl, tetrahydropyranyl, 3,4,5,6-tetrahydro-pyridinyl, 1,2,3,4-tetrahydropyrimidinyl, 3,4,5,6-tetrahydropyrimidinyl, thiadiazolyl, thiazolidinyl, thiazolyl, thienyl, thieno[5,1-c]pyridinyl, thiochromanyl, triazolyl, 1,3,4-triazolo[2,3-b]pyrimidinyl, xanthenyl and the like.

Values of Het³ that may be mentioned include pyridinyl (e.g. pyridin-2-yl).

Substituents on heterocyclic (Het¹ to Het¹⁵ and Het^{a} to Het^{f}) groups may, where appropriate, be located on any atom in the ring system including a heteroatom. The point of attachment of heterocyclic (Het¹ to Het¹⁵ and Het^{e} to Het^{f}) groups may be via any atom in the ring system including (where appropriate) a heteroatom, or an atom on any fused carbocyclic ring that may be present as part of the ring system.

For the avoidance of doubt, cycloalkyl and cycloalkenyl groups may be monocyclic or, where the number of C-atoms allows, be bi- or tri-cyclic (although monocyclic cycloalkyl and cycloalkenyl are particular embodiments that may be mentioned). Further, when a cycloalkyl or cycloalkenyl group is fused to two phenyl groups, the phenyl groups may also be fused to each other (to form a fused tricyclic ring system).

Compounds of formula I may exhibit tautomerism. All tautomeric forms and mixtures thereof are included within the scope of the invention.

Compounds of formula I may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation, or by derivatisation, for example with a homochiral acid followed by separation of the diastereomeric esters by conventional means (e.g. HPLC, chromatography over silica). All stereoisomers are included within the scope of the invention.

Abbreviations are listed at the end of this specification. The wavy lines on the bonds in structural fragments signify the bond positions of those fragments.

Particular values that may be mentioned in relation to compounds of formula I include those in which:
(1) A represents C₁₋₄ alkylene;
(2) R¹ represents
   (a) C₁₋₆ alkyl, C₂₋₆ alkenyl, C₂₋₆ alkynyl (which latter three groups are optionally substituted by one or more substituents selected from halo, CN, C₃₋₈ cycloalkyl (optionally substituted by one or more substituents selected from halo, OH, ═O, C₁₋₆ alkyl, C₁₋₆ alkoxy and aryl), OR^{9a}, SR^{9b}, S(O)₂R^{9b}, S(O)₂N(H)R^{9c}, N(H)S(O)₂R^{9f}, N(R^{9b})(R^{9h}), C(O)R⁹ⁱ, OC(O)R⁹ⁱ, C(O)OR⁹ⁱ, N(H)C(O)R⁹ⁱ, C(O)N(H)R⁹ⁱ, aryl and Het¹),
   (b) C₃₋₈ cycloalkyl or C₄₋₈ cycloalkenyl, which latter two groups are optionally fused to one or two phenyl groups and are optionally substituted by one or more substituents selected from halo, ═O, C₁₋₆ alkyl, C₄₋₆ cycloalkyl (optionally substituted by one or more substituents selected from halo, C₁₋₄ alkyl, C₁₋₄ alkoxy and phenyl), OR^{9a}, SR^{9b}, S(O)₂R^{9b}, S(O)N(H)R^{9c}, N(H)S(O)R^{9f}, N(R^{9g})(R^{9h}), OC(O)R⁹ⁱ, C(O)OR⁹ⁱ, N(H)C(O)R⁹ⁱ, C(O)N(H)R⁹ⁱ, aryl and Het²,
   (c) aryl, or
   (d) Het³;
(3) R⁹ⁱ to R⁹ⁱ independently represent, at each occurrence,
   (a) H,
   (b) C₁₋₆ alkyl, C₂₋₆ alkenyl, C₂₋₆ alkynyl (which latter three groups are optionally substituted by one or more substituents selected from halo, OH, C₁₋₄ alkoxy, aryl and Het⁴),
   (c) C₄₋₆ cycloalkyl, C₄₋₆ cycloalkenyl (which latter two groups are optionally substituted by one or more substituents selected from halo, ═O and C₁₋₄ alkyl),
   (d) aryl or
   (e) Het⁶,
   provided that R^{9b} does not represent H when n is 1 or 2;
(4) R^{2a} and R^{2b} both represent H, both represent methyl or both represent F;
(5) R^{3a} and R^{3b} both represent H, both represent methyl or both represent F;
(6) R⁴ represents C₁₋₄ alkyl (which latter group is optionally substituted by one or more halo substituents);
(7) R^{5a} and R^{5b} independently represent H or F;
(8) R⁶ represents H;
(9) G represents C₁₋₃ alkylene;
(10) R⁷ and R⁸ independently represent C₁₋₂ alkyl optionally substituted by OR¹⁰, provided that at least one of R⁷ and R⁸ is substituted by OR¹⁰;
(11) R¹⁰ represents H or —C(O)—X—R¹¹;
(12) X represents O or, particularly, a direct bond;
(13) R¹¹ represents
   (a) C₁₋₆ alkyl optionally substituted by one or more substituents selected from halo, C₃₋₆ cycloalkyl, C₅₋₆ cycloalkenyl (which latter two groups are optionally substituted by one or more substituents selected from halo and methyl), aryl and Het⁷,
   (b) C₃₋₆ cycloalkyl, C₅₋₆ cycloalkenyl (which latter two groups are optionally substituted by one or more substituents selected from halo and methyl),
   (c) aryl or
   (d) Het⁹;
(14) R^{12a} to R^{12d} independently represent H or, particularly, C₁₋₄ alkyl (such as methyl or ethyl);
(15) each aryl independently represents phenyl optionally substituted by one or more substituents selected from
   (a) halo,
   (b) CN,
   (c) C₁₋₈ alkyl, C₂₋₄ alkenyl, C₂₋₄ alkynyl (which latter three groups are optionally substituted by one or more substituents selected from halo, OH, C₁₋₂ alkoxy, C(O)OH, C(O)O—C₁₋₂ alkyl and phenyl),
   (d) C₃₋₆ cycloalkyl optionally substituted by one or more substituents selected from halo, ═O and C₁₋₄ alkyl,
   (e) OR^{13a}
   (f) SR^{13b}, S(O)₂R^{13b}
   (g) S(O)₂N(H)R^{13c},
   (h) N(H)S(O)₂R^{13f},
   (i) N(H)R^{13g},
   (j) C(O)R¹³ⁱ, C(O)OR³¹, OC(O)R¹³ⁱ, C(O)N(H)R¹³ⁱ, N(H)C(O)R¹³ⁱ, N(H)C(O)OR¹³ⁱ,
   (k) phenyl (which latter group is optionally substituted by one or more halo atoms),
   (l) Het¹² and
   (m) Si(CH₃)₃;
(16) R^{13a} to R¹³ⁱ independently represent, at each occurrence,
   (a) H,
   (b) C₁₋₈ alkyl optionally substituted by one or more substituents selected from halo, OH, C₁₋₂ alkoxy, phenyl (which latter group is optionally substituted by one or more halo atoms) and Het¹³ (e.g. one or more substituents selected from halo, OH, C₁₋₂ alkoxy and phenyl (which latter group is optionally substituted by one or more halo atoms)),
   (c) C₃₋₆ cycloalkyl optionally substituted by one or more substituents selected from halo, ═O and C₁₋₄ alkyl,
   (d) phenyl optionally substituted by one or more halo atoms or
   (e) Het¹⁵,
   provided that R^{13b} does not represent H;
(17) Het¹ to Het¹⁵ independently represent 5- to 13-membered heterocyclic groups containing one to four heteroatoms selected from oxygen, nitrogen and/or sulfur, which heterocyclic groups may comprise one, two or three rings and may be substituted by one or more substituents selected from
   (a) halo,
   (b) CN,
   (c) C₁₋₈ alkyl, C₂₋₄ alkenyl, C₂₋₄ alkynyl (which latter three groups are optionally substituted by one or more substituents selected from halo, OH and C₁₋₂ alkoxy),
   (d) C₃₋₆ cycloalkyl optionally substituted by one or more substituents selected from halo, ═O and C₁₋₄ alkyl,
   (e) ═O,
   (f) OR^{15a},
   (g) S(O)₂R^{15b},
   (h) S(O)₂N(O)R^{15c},
   (i) N(H)S(O)₂R^{15f},
   (j) N(H)R^{15g},
   (j) C(O)R¹⁵ⁱ, C(O)OR¹⁵ⁱ, C(O)N(DR¹⁵ⁱ, N(H)C(O)R¹⁵ⁱ, N(H)C(O)OR¹⁵ⁱ,
   (l) phenyl (which latter group is optionally substituted by halo) and
   (m) Het^{c};
(18) R^{15a} to R¹⁵ⁱ independently represent, at each occurrence,
   (a) H,
   (b) C₁₋₆ alkyl optionally substituted by one or more substituents selected from halo, OH, C₁₋₂ alkoxy and phenyl,
   (c) C₃₋₆ cycloalkyl optionally substituted by one or more substituents selected from halo, ═O and C₁₋₄ alkyl,
   (d) phenyl optionally substituted by halo or
   (e) Het^{f},
   provided that R^{15b} does not represent H;
(19) Het^{a} to Het^{f} independently represent 5- or 6-membered heterocyclic groups containing, as heteroatoms, one oxygen or sulfur atom and/or one to three nitrogen atoms, which heterocyclic groups may be substituted by one or more substituents selected from halo and C₁₋₄ alkyl.

Compounds of formula I that may be mentioned include those in which R^{5a} and R^{5b} both take the same definition (i.e. compounds in which R^{5g} and R^{5b} both represent H, both represent F or both represent methyl, CH₂F, CHF₂ or CF₃). Another embodiment of the invention relates to compounds of formula I in which

A represents C(O) or C(O)NH (in which latter group the NH moiety is attached to R¹) and R¹ represents:
(a) C₁₋₆ alkyl, C₂₋₆ alkenyl, C₂₋₆ alkynyl, which latter three groups are
   (i) substituted by one substituent selected from C₃₋₈ cycloalkyl (optionally substituted by one or more substituents selected from halo, OH, ═O, C₁₋₆ alkyl, C₁₋₆ alkoxy and aryl), aryl and Het¹, and
   (ii) optionally substituted by one or more further substituents selected from halo, CN, C₄₋₆ cycloalkyl (optionally substituted by one or more substituents selected from halo and C₁₋₄ alkyl), OR^{9a}, SR^{9b}, S(O)₂R^{9b}, S(O)₂N(H)R^{9c}, N(H)S(O)₂R^{9f}, N(R^{9g})(R^{9h}), OC(O)R⁹ⁱ, C(O)OR⁹ⁱ, N(H)C(O)R⁹ⁱ, C(O)N(H)R⁹ⁱ, aryl and Het¹;
(b) C₃₋₈ cycloalkyl or C₄₋₈ cycloalkenyl, which latter two groups are
   (i) fused to one or two phenyl groups and optionally substituted by one or more substituents selected from halo, C₁₋₄ alkyl and C(O)OR⁹ⁱ, or
   (ii) substituted by aryl and optionally further substituted by one or more substituents selected from halo and C₁₋₄ alkyl;
(c) aryl; or
(d) Het³,
   wherein R^{9a} to R^{9c}, R^{9f} to R⁹ⁱ aryl, Het¹ and Het³ are as defined above or below.

Yet another embodiment of the invention relates to compounds of formula I in which A represents S(O)₂ and R¹ represents:
(a) C₁₋₃ alkyl or Q-3 alkenyl, which latter two groups are substituted by aryl and are optionally further substituted by one or more halo atoms;
(b) C₁₋₆ alkyl optionally substituted by one or more substituents selected from halo, OR^{9a} and S(O)₂R^{9b};
(c) C₃₋₆ monocyclic cycloalkyl optionally substituted by one or more substituents selected from halo and C₁₋₄ alkyl;
(d) C₆₋₈ bicyclic cycloalkyl optionally substituted by one or more substituents selected from halo, ═O and C₁₋₆ alkyl;
(c) aryl; or
(d) Het³,
   wherein R^{6a}, R^{6b} and Het³ are as defined above or below.

In a still further embodiment of the invention relates to compounds of formula I in which A represents C₁₋₆ alkylene and R¹ represents:
(a) C₁₋₆ alkyl or C₂₋₆ alkenyl, which latter two groups are optionally substituted by one or more substituents selected from halo and OH;
(b) C₃₋₈ cycloalkyl or C₄₋₈ (e.g. C₄₋₆) cycloalkenyl, which latter two groups are optionally substituted by one to four substituents selected from halo, ═O, OH, C₁₋₄ alkyl, O—C₁₋₄ alkyl (which latter two groups are optionally substituted by one or more halo (e.g. F) atoms) and aryl, or, particularly,
(c) aryl (e.g. naphthyl or, particularly, phenyl), or
(d) Het³,
   wherein Het³ is as defined above or below.

More particular values that may be mentioned in relation to compounds of formula

I include those in which:
(1) A represents C₁₋₃ alkylene;
(2) R¹ represents
   (a) C₁₋₅ alkyl, C₂₋₄ alkenyl (which latter two groups are optionally substituted by one or more substituents selected from halo, C₆₋₈ bicyclic cycloalkyl, C₃₋₆ monocyclic cycloalkyl (which latter two groups are optionally substituted by one or more substituents selected from halo, ═O, C₁₋₄ alkyl, C₁₋₄ alkoxy and phenyl (which latter group is optionally substituted by one or more substituents selected from halo, C₁₋₄ alkyl and C₁₋₄ alkoxy)), OR^{9a}, SR^{9b}, S(O)₂R^{9b}, C(O)R⁹ⁱ, OC(O)R⁹ⁱ, C(O)OR⁹ⁱ, aryl and Het¹),
   (b) C₃₋₆ cycloalkyl or C₄₋₈ (e.g. C₄₋₆) cycloalkenyl, which latter two groups are optionally fused to one or two phenyl groups and are optionally substituted by one or more substituents selected from halo, ═O, C₁₋₄ alkyl, OR^{9a}, C(O)OR⁹ⁱ and phenyl (which latter group is optionally substituted by one or more substituents selected from halo, C₁₋₄ alkyl and C₁₋₄ alkoxy),
   (c) aryl, or
   (d) Het³;
(3) R^{9a} to R⁹ⁱ independently represent, at each occurrence,
   (a) H,
   (b) C₁₋₆ alkyl, C₂₋₄ alkenyl (which latter two groups are optionally substituted by one or more substituents selected from halo, OH, C₁₋₄ alkoxy and phenyl),
   (c) C₄₋₆ cycloalkyl (which latter group is optionally substituted by one or more substituents selected from halo and C₁₋₂ alkyl) or
   (d) phenyl (which latter group is optionally substituted by one or more substituents selected from halo, C₁₋₄ alkyl and C₁₋₄ alkoxy)
   provided that R^{9b} does not represent H;
(4) R^{2a} and R^{2b} both represent H;
(5) R^{3a} and R^{3b} both represent H;
(6) R⁴ represents C₁₋₃ alkyl optionally substituted by one or more F atoms;
(7) R^{5a} and R^{1b} both represent H or both represent F;
(8) G represents C₁₋₃ n-alkylene;
(9) R⁷ and R⁸ independently represent methyl optionally substituted by OR¹⁰, provided that at least one of R⁷ and R⁸ is substituted by OR¹⁰;
(10) R¹⁰ represents H or —C(O)R¹¹
(11) R¹¹ represents
   (a) C₁₋₄ alkyl optionally substituted by one or more substituents selected from halo, Q-6 cycloalkyl, phenyl (which latter group is optionally substituted by one or more substituents selected from halo and methyl) and Het⁷,
   (b) C₅₋₆ cycloalkyl optionally substituted by one or more substituents selected from chloro, fluoro and methyl,
   (c) aryl or
   (d) Het⁹;
(12) each aryl independently represents phenyl or naphthyl, each of which groups may be substituted by one or more substituents selected from
   (a) F, Cl, Br,
   (b) CN,
   (c) C₁₋₆ alkyl, C₂₋₃ alkenyl (which latter two groups are optionally substituted by one or more substituents selected from F, Cl, C(O)OH, C(O)OCH₃ and phenyl),
   (d) C₃₋₅ cycloalkyl,
   (e) OR^{13a},
   (f) S—C₁₋₂ alkyl, S(O)₂—C₁₋₂ alkyl (the alkyl parts of which latter two groups are optionally substituted by one or more F atoms),
   (g) S(O)₂NH₂, S(ON(H)CH₃,
   (h) N(H)S(O—C₁₋₂ alkyl (the alkyl part of which latter group is optionally substituted by one or more F atoms),
   (i) NH₂, N(H)C₁₋₂ alkyl,

(j) CHO, C(O)—C₁₋₄ alkyl (the alkyl part of which latter group is optionally substituted by one or more F or Cl atoms), C(O)OH, C(O)O—C₁₋₄ alkyl, C(O)NH₂, C(O)N(H)—C₁₋₄ alkyl, N(H)C(O)—C₁₋₄ alkyl, N(H)C(O)O—C₁₋₄ alkyl,
(k) phenyl (which latter group is optionally substituted by one to four substituents selected from F, Cl and Br),
(l) Het¹² and
(m) Si(CH₃)₃;
(13) R^{13a} represents
   (a) H,
   (b) C₁₋₅ alkyl optionally substituted by phenyl or one or more substituents selected from F, Cl and Het¹³,
   (c) C₃₋₅ cycloalkyl or
   (d) phenyl optionally substituted by one to four substituents selected from F, Cl and Br;
(14) Het¹ represents a 5- to 10-membered heterocyclic group containing one to three heteroatoms selected from oxygen, nitrogen and/or sulfur, which heterocyclic group may comprise one or two rings and may be substituted by one to three substituents selected from F, Cl, Br, C₁₋₄ alkyl, ═O and OH;
(15) Het³, Het⁷ and Het⁹ independently represent 5- to 13-membered heterocyclic groups containing one to four heteroatoms selected from oxygen, nitrogen and/or sulfur, which heterocyclic groups may comprise one, two or three rings and may be substituted by one to four substituents selected from
   (a) F, Cl, Br,
   (b) C₁₋₄ alkyl (which latter group is optionally substituted by one or more substituents selected from F, Cl and OH),
   (c) C₃₋₅ cycloalkyl,
   (d) ═O,
   (e) OH, O—C₁₋₂ alkyl (which latter group is optionally substituted by one or more substituents selected from F and Cl),
   (g) S(O)₂—C₁₋₂ alkyl (which latter group is optionally substituted by one or more F atoms), S(O)-phenyl (the phenyl part of which latter group is optionally substituted by one to four substituents selected from F, Cl, Br, methyl and methoxy),
   (h) S(O)₂NH₂, S(ON(H)—C₁₋₂ alkyl,
   (i) N(H)S(O)₂—C₁₋₂ alkyl,
   (j) NH₂, N(H)—C₁₋₂ alkyl,
   (j) C(O)—C₁₋₄ alkyl, C(O)-phenyl (the phenyl part of which latter group is optionally substituted by one to four substituents selected from F, Cl, Br, methyl and methoxy), C(O)OH, C(O)O—C₁₋₄ alkyl, C(O)NH₂, C(O)N(H)—Cl₄ alkyl, N(H)C(O)—C₁₋₄ alkyl, N(H)C(O)O—C₁₋₄ alkyd
   (l) phenyl (which latter group is optionally substituted by one to four substituents selected from F, Cl and Br) and
   (m) Het^{c};
(16) Het¹² represents 5- or 6-membered monocyclic heterocyclic group containing, as heteroatom(s), one sulfur or oxygen atom and/or one to three nitrogen atoms, which heterocyclic groups may comprise one, two or three rings and may be substituted by one or more substituents selected F, Cl, Br, C₁₋₄ alkyl, ═O and OH;
(17) Het^{c} represents a 5- or 6-membered heterocyclic group containing, as heteroatoms, one oxygen atom and/or one or two nitrogen atoms, which heterocyclic groups may be substituted by one or more substituents selected from F, Cl, Br and methyl.

Yet more particular values that may be mentioned in relation to compounds of formula I include those in which:

A represents C₁₋₃ alkylene optionally substituted by one or more F atoms;
R¹ represents
(a) C₁₋₃ alkyl substituted by phenyl (which latter group is optionally substituted by one or more substituents selected from halo, C₁₋₄ alkyl and C₁₋₄ alkoxy (which latter two groups are optionally substituted by one or more F atoms)),
(b) phenyl or naphthyl (which latter two groups are optionally substituted by one or more substituents selected from CN, halo, C₁₋₄ alkyl, C₁₋₄ alkoxy (which latter two groups are optionally substituted by one or more F atoms), O-phenyl, OCH₂-Het¹³ and Het¹²,
(c) a 5- or 6-membered monocyclic (e.g. aromatic) heterocyclic group containing, as heteroatom(s), an oxygen or sulfur atom and/or one to three nitrogen atoms, which heterocyclic group is optionally substituted by one to four substituents selected from F, Cl, Br, ═O, OH, C₁₋₄ alkyl (which latter group is optionally substituted by one or more halo atoms or by OH), C₁₋₄ alkoxy, S(O)₂-phenyl, C(O)-phenyl, phenyl and Het^{c},
(d) a 9- or 10-membered bicyclic (e.g. part-aromatic) heterocyclic group containing one to three heteroatoms selected from oxygen, nitrogen and/or sulfur (e.g. two oxygen atoms), which heterocyclic group is optionally substituted by one to four substituents selected from F, Cl, Br, C₁₋₄ alkyl and C₁₋₄ alkoxy,
(e) C₁₋₅ alkyl, or
(f) C₄₋₇ cycloalkyl or Q-7 cycloalkenyl, which latter two groups are optionally substituted by one or more methyl groups;
   Het¹² represents a 5- or 6-membered monocyclic heterocyclic group containing, as heteroatom(s), one sulfur or oxygen atom and/or one or two nitrogen atoms, which heterocyclic group may be substituted by one to three substituents selected from F, Cl and methyl;
   Het¹³ represents a 5- or 6-membered monocyclic, aromatic heterocyclic group containing, as heteroatom(s), one sulfur or oxygen atom and/or one or two nitrogen atoms, which heterocyclic group may be substituted by one to three substituents selected from F, Cl, methyl and methoxy;
   Het^{c} represents a 5- or 6-membered monocyclic heterocyclic group containing, as heteroatom(s), an oxygen or sulfur atom and/or or one or two nitrogen atoms, which heterocyclic group is optionally substituted by one to four substituents selected from F, Cl, Br, C₁₋₄ alkyl and C₁₋₄ alkoxy;
   R^{2a}, R^{2b}, R^{3a}, R^{3b} all represent H;
   R⁴ represents methyl optionally substituted by one or more F atoms;
   R^{5a} and R^{5b} both represent H;
   G represents CH₂ or (CH₂)₂;
   R⁷ represents CH₂OR¹⁰;
   R⁸ represents methyl;
   R¹¹ represents C₁₋₄ alkyl (optionally substituted by one or more halo atoms) or phenyl (which latter group is optionally substituted by one or more substituents selected from halo, methyl and methoxy).

Still more particular values that may be mentioned in relation to compounds of formula I include those in which:

A represents C₁₋₃ (e.g. C₁₋₂) alkylene (optionally gem-disubstituted by two F atoms);
R¹ represents
(a) C₁₋₂ alkyl substituted by phenyl (which latter group is optionally substituted by one or more substituents selected from F, Cl and Br), or
(b) phenyl (which latter group is optionally substituted by one or more substituents selected from F, Cl, Br, CN, Q-3 alkyl, C 3 alkoxy (which latter group two groups are optionally substituted by one or more F atoms (thus forming, for example, C₁₋₂ alkyl, CF₃, C₁₋₂ alkoxy or OCF₃)), O-phenyl, O—CH₂-Het¹³ and Het¹²),
(c) naphthyl (e.g. 1-naphthyl), or
(d) pyridinyl (e.g. pyridin-2-yl or pyridin-3-yl) optionally substituted by one or two substituents selected from F, Cl, (N-)oxo, OH, Cl₄ alkyl (such as methyl, which C₁₋₄ alkyl group is optionally substituted by one or more halo atoms or by OH) or, particularly, C₁₋₄ alkoxy (e.g. tert-butoxy or methoxy) or Het^{c},
(e) pyridonyl (e.g. 2-pyridin-3-yl) optionally substituted by one or two substituents selected from F, Cl, and C₁₋₄ alkyl (e.g. methyl);
(f) pyrazinyl (e.g. pyrazin-2-yl) optionally substituted by one or two substituents selected from F, Cl and methyl;
(g) a 5-membered aromatic heterocyclic group containing, as heteroatom(s), an oxygen or sulfur atom and/or one to three nitrogen atoms (e.g. imidazolyl, isoxazolyl, pyrazolyl, pyrrolyl, thiazolyl, or thienyl), which heterocyclic group is optionally substituted by one to four (e.g. one to three) substituents selected from F, Cl, C₁₋₄ alkyl (e.g. methyl or ethyl), C₁₋₄ alkoxy (e.g. methoxy), S(O)₂-phenyl, C(O)-phenyl, phenyl, morpholinyl (e.g. morpholin-4-yl), 1,3,4-triazolyl (e.g. 1,3,4-triazol-1-yl), thienyl (e.g. 2-thienyl) and pyridinyl (e.g. pyridin-2-yl),
(h) 2,3-dihydrobenzofuranyl, benzomorpholinyl, benzodioxanyl, 2,1,3-benzoxadiazolyl, or, particularly, benzodioxolyl or quinolinyl, all of which groups are optionally substituted by one or more (e.g. one to three) substituents selected from F, Cl, C₁₋₂ alkyl and C₁₋₂ alkoxy,
(i) C₁₋₄ alkyl (e.g. isopropyl or tert-butyl), or
(j) cyclopentyl, cyclohexyl or C₇ bicyclic cycloalkenyl (e.g. bicyclo[2.2.1]heptene, which latter three groups are optionally substituted by one to four methyl groups;
   Het¹² represents a 6-membered, saturated, monocyclic heterocyclic group containing, as heteroatom(s), one oxygen atom and/or one or two nitrogen atoms, which heterocyclic group may be substituted by one or two methyl substituents;
   Het¹³ represents a 5-membered, monocyclic, aromatic heterocyclic group containing, as heteroatom(s), one sulfur or oxygen atom and/or one or two nitrogen atoms, which heterocyclic group may be substituted by one to three substituents selected from Cl and methyl;
   Het^{c} represents a 6-membered, saturated, monocyclic heterocyclic group containing, as heteroatom(s), one oxygen atom and/or one or two nitrogen atoms, which heterocyclic group may be substituted by one or two methyl substituents.

Other particular values that may be mentioned in relation to compounds of formula I include those in which:

A represents CH(CH₃)CH₂ (in which latter group the CH(CH₃) unit is attached to R¹) or, particularly, CH₂, (CH₂)₂ or CF₂CH₂ (in which latter group the CF₂ unit is attached to R¹);
R¹ represents
(a) isopropyl or tert-butyl,
(b) cyclopentyl, cyclohexyl or bicyclo[2.2.1]hept-5-ene,
(c) phenyl optionally substituted by one or two substituents selected from halo (e.g. F or Cl), CN, methyl, CF₃, methoxy or OCF₃,
(d) imidazolyl optionally substituted by one to three substituents selected from halo (e.g. F or Cl) and methyl,
(e) isoxazolyl (e.g. isoxazol-3-yl or isoxazo-4-yl) optionally substituted by one or two methyl groups,
(f) thiazolyl (e.g. thiazol-5-yl) optionally substituted by one or two methyl groups,
(g) thienyl (e.g. thien-2-yl) optionally substituted by halo (e.g. F or Cl),
(h) pyrazolyl (e.g. pyrazol-4-yl) optionally substituted by one to three substituents selected from halo (e.g. F or Cl), methyl and ethyl,
(i) pyrrolyl (e.g. pyrrol-2-yl or pyrrol-3-yl) optionally substituted by one to three methyl groups,
(j) pyridinyl (e.g. pyridin-2-yl or pyridin-3-yl) optionally substituted by halo (e.g. F or Cl) or methyl, and optionally in the form of an N-oxide,
(k) pyridonyl (e.g. 2-pyridin-3-yl),
(l) pyrazinyl (e.g. pyrazin-2-yl),
(m) benzodioxolyl (e.g. 5-benzodioxolyl) optionally substituted by halo (e.g. Cl),
(n) benzomorpholinyl (e.g. 7-benzomorpholinyl) optionally substituted by methyl;
(o) 2,1,3-benzoxadiazolyl (e.g. 2,1,3-benzoxadiazol-5-yl),
(p) 2,3-dihydrobenzofuranyl (e.g. 2,3-dihydrobenzofuran-5-yl) or
(q) quinolinyl (e.g. 8-quinolinyl);

In another embodiment of the invention, the compound of formula I is a compound of formula Ia,

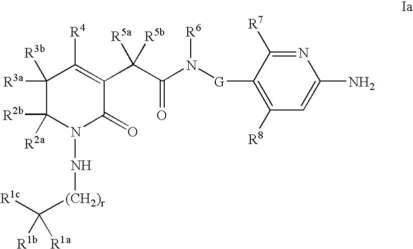

wherein:
R^{1a} represents aryl or Het³;
R^{1b} and R^{1c} independently represent H, halo or methyl;
r represents 0 or 1; and
R^{2a}, R^{2b}, R^{3a}, R^{3b}, R⁴, R^{5a}, R^{5b}, R⁶ to R⁸, G, aryl and Het³ are as hereinbefore defined.

Particular values that may be mentioned in relation to compounds of formula Ia include those in which:

R^{1b} and R^{1c} either both represent H or, when r represents 1, both represent F;
R^{2a} and R^{2b} both represent H;
R^{3a} and R^{3b} both represent H;
R⁴ represents methyl;
R^{5a} and R^{5b} both represent H;
R⁶ represents H;
G represents C₁₋₂ n-alkylene (e.g. CH₂).

More particular values that may be mentioned in relation to compounds of formula Ia include those in which:

R^{1a} represents phenyl (optionally substituted by one or more substituents selected from halo (e.g. F or Cl), C₁₋₃ alkyl (e.g. methyl) and C₁₋₃ alkoxy (e.g. methoxy) (which alkyl and alkoxy groups are optionally substituted by one or more F atoms)) or Het³;
R^{1b} and R^{1c} both represent F;
r represents 1;
Het³ represents a 5- or 6-membered heterocycle containing, as heteroatom(s), one oxygen or sulfur atom and/or one or two nitrogen atoms, which heterocyclic group may be substituted by one or more substituents selected from halo (e.g. Cl), C₁₋₃ alkyl (e.g. methyl) and C₁₋₃ alkoxy (e.g. methoxy), which alkyl and alkoxy groups are optionally substituted by one or more F atoms;
R⁷ represents CH₂OR¹⁰;
R⁸ represents methyl;
R¹¹ represents C₁₋₂ alkyl (optionally substituted by one or more Cl or F atoms) or phenyl (which latter group is optionally substituted by one or more substituents selected from Cl, F and methyl).

For the avoidance of doubt, the particular definitions of groups given above in relation to compounds of formula Ia are also, where relevant, particular definitions of the equivalent groups in compounds of formula I (e.g. definitions of the group R^{1a} may be viewed as particular definitions of the group R¹). Moreover, references herein to compounds of formula I also include, where relevant, references to compounds of formula Ia.

One embodiment of the invention relates to compounds of formulae I and Ia in which R¹⁰ represents H. However, another embodiment of the invention relates to compounds of formulae I and Ia in which R¹⁰ represents —C(O)—X—R¹¹.

A still further embodiment of the invention relates to compounds of formulae I and Ia in which R⁷ is substituted by OR¹⁰ and R⁸ is not so substituted.

Particular embodiments of the invention that may be mentioned include the compounds of the Examples disclosed hereinafter. In this respect, compounds of the invention that may be mentioned include:
N-{[6-amino-2-(hydroxymethyl)-4-methylpyridin-3-yl]methyl}-2-{1-[(2,2-difluoro-2-pyridin-2-ylethyl)amino]-4-methyl-2-oxo-1,2,5,6-tetrahydropyridin-3-yl}acetamide;
(6-amino-3-{[({1-[(2,2-difluoro-2-pyridin-2-ylethyl)amino]-4-methyl-2-oxo-1,2,5,6-tetrahydropyridin-3-yl}acetyl)amino]methyl}-4-methylpyridin-2-yl)methyl acetate; and
(6-amino-3-{[({1-[(2,2-difluoro-2-pyridin-2-ylethyl)amino]-4-methyl-2-oxo-1,2,5,6-tetrahydropyridin-3-yl}acetyl)amino]methyl}-4-methylpyridin-2-yl)methyl benzoate.

### Preparation

Compounds of formula I (including compounds of formula Ia) may be made in accordance with techniques well known to those skilled in the art, for example as described hereinafter.

According to a further aspect of the invention there is provided a process for the preparation of a compound of formula I, which comprises:

(a) coupling of a compound of formula II,

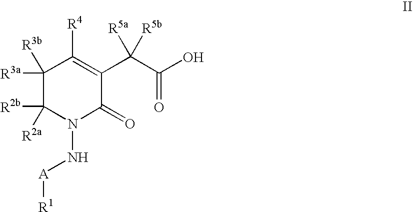

wherein R¹, R^{2a}, R^{2b}, R^{3a}, R^{3b}, R⁴, R^{5a}, R^{5b} and A are as hereinbefore defined, with a compound of formula III,

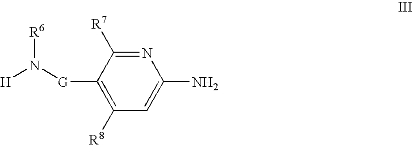

or a derivative thereof that is protected at the 2-amino substituent of the pyridine ring, wherein R⁶ to R⁸ and G are as hereinbefore defined, for example in the presence of a coupling agent (e.g. oxalyl chloride in DMF, EDC, DCC, HBTU, HATU, PyBOP, HOBt or TBTU), an appropriate base (e.g. pyridine, DMAP, TEA, 2,4,6-collidine or DIPEA) and a suitable organic solvent (e.g. DCM, MeCN, EtOAc or DMF);
(b) reaction of a compound of formula IV,

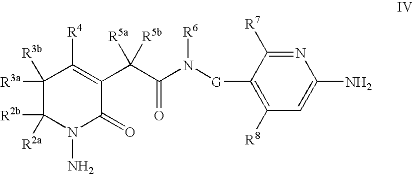

or a derivative thereof that is protected at the 2-amino substituent of the pyridine ring, wherein R^{2a}, R^{2b}, R^{3a}, R^{3b}, R⁴, R^{5a}, R^{5b}, R⁶ to R⁸ and G are as hereinbefore defined, with a compound of formula V,

R¹-A-Lg¹ V

wherein Lg¹ represents a suitable leaving group (e.g. halo, trifluoromethanesulfonate or OH) and R¹ and A are as hereinbefore defined, for example under conditions known to those skilled in the art (such as in the presence of an appropriate base (e.g. K₂CO₃, pyridine or 2,6-di-tert-butyl-4-methylpyridine) and a suitable solvent (e.g. DCM or 1,2-dichloroethane));
(c) for compounds of formula I in which A represents C(O)NH, reaction of a compound of formula IV, as hereinbefore defined, or a derivative thereof that is protected at the 2-amino substituent of the pyridine ring, with a compound of formula VI,

R¹—N═C═O VI

wherein R¹ is as hereinbefore defined, for example under conditions known to those skilled in the art (such as at ambient temperature (e.g. 15 to 25° C.) in the presence of a suitable solvent (e.g. DCM));
(d) for compounds of formula I in which A represents C₁₋₆ alkylene, reaction of a compound of formula IV, as hereinbefore defined, or a derivative thereof that is protected at the 2-amino substituent of the pyridine ring, with a compound of formula VII,

R¹—C₀₋₅ alkylene-CHO VII

wherein R¹ is as hereinbefore defined, for example under conditions known b those skilled in the art (such as at reflux in the presence of a suitable solvent (e.g. ethanol), followed by reduction in the presence of a reducing agent (e.g. NaBH₃CN), for example under conditions known to those skilled in the art (e.g. at ambient temperature (such as 15 to 25° C.) in the presence of a suitable solvent (such as ethanol); or
(e) for compounds of formula I in which R⁷ and/or R⁸ represents C₁₋₄ alkyl substituted by —O—C(O)—X—R¹¹, reaction of a corresponding compound of formula I in which R⁷ and/or R⁸ represents C₁₋₄ alkyl substituted by —OH with a compound of formula VIII,

R¹¹—X—C(O)-Lg² VIII

wherein Lg² represents a suitable leaving group (e.g. halo or, when X represents a direct bond, OH or OC(O)R¹¹) and R¹¹ and X are as hereinbefore defined, for example under conditions known to those skilled in the art (such as reaction in the presence of an appropriate solvent (e.g. DCM, MeCN, EtOAc or DMF) and optionally in the presence of a suitable base (e.g. TEA or pyridine) and/or, when X represents a direct bond and Le represents OH, a coupling agent (e.g. oxalyl chloride in DMF, EDC, DCC, HBTU, HATU, PyBOP or TBTU)).

Compounds of formula II may be prepared by hydrolysis of a compound of formula IX,

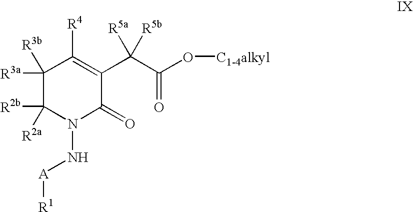

wherein R¹, R^{2a}, R^{2b}, R^{3a}, R^{3b}, R⁴, R^{5a}, R^{5b} and A are as hereinbefore defined, for example under conditions known to those skilled in the art (e.g. by basic hydrolysis in the presence of an alkali metal hydroxide (e.g. NaOH or, particularly, LiOH) and a suitable solvent (e.g. water, THF or a mixture thereof)).
Compounds of formula III in which R⁷ and/or R¹ represents C₁₋₄ alkyl substituted by OH may be prepared by hydrolysis of a corresponding compound of formula III in which R⁷ and/or R⁸ (as appropriate) represents C₁₋₄ alkyl substituted by OC(O)R¹¹, for example under conditions know to those skilled in the art (such as hydrolysis under conditions analogous to those described above in respect of the preparation of compounds of formula II).

Compounds of formula III in which R⁷ represents CH₂OC(O)R¹¹ and R⁸ represents C₁₋₄ alkyl may be prepared by reaction of a corresponding compound of formula X,

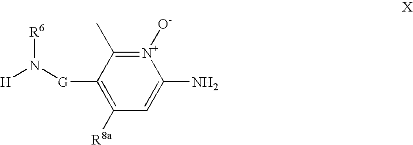

or an N-protected or N,N′-diprotected derivative thereof, wherein R^{8a} represents C₁₋₄ alkyl and R⁶ is as hereinbefore defined, with a compound of formula XI,

[R¹¹C(O)]₂O XI

wherein R¹¹ is as hereinbefore defined, followed by reaction with an amine base (e.g. a primary amine or, particularly, an N,N-dialkylated alkylenediamine such as N,N-diethylethylenediamine), for example under conditions known to those skilled in the art (such as reaction with the compound of formula XI at elevated temperature (e.g. 50 to 80° C.), followed by reaction with the amine base at ambient temperature, optionally in the presence of a suitable solvent (e.g. MeCN)).

Compounds of formula IV may be prepared by reduction of a compound of formula XI,

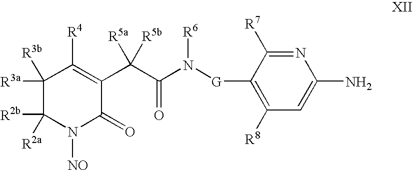

or a derivative thereof that is protected at the 2-amino substituent of the pyridine ring, wherein R^{2a}, R^{2b}, R^{3a}, R^{3b}, R⁴, R^{5a}, R^{5b}, R⁶ to R⁸ and G are as hereinbefore defined, for example under conditions that are well known to those skilled in the art (such as by reaction with zinc metal (e.g. zinc powder or iron metal powder) in the presence of an appropriate acid (e.g. acetic acid or hydrochloric acid) and optionally in the presence of a suitable solvent (e.g. methanol)).

Compounds of formula VII may be prepared by oxidation of an alcohol of formula XIII,

R¹—C₀₋₅ alkylene-CH₂OH XIII

wherein R¹ is as hereinbefore defined, for example under conditions known to those skilled in the art, such as reaction with PCC, oxalyl chloride and DMSO (Swern oxidation) or, particularly, Dess-Martin periodinane in the presence of a suitable solvent (such as DCM).

Compounds of formula IX may be prepared by reaction of a compound of formula XIV,

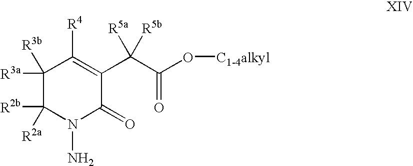

wherein R^{2a}, R^{2b}, R^{3a}, R^{3b}, R⁴, R^{5a} and R^{5b} are as hereinbefore defined, with a compound of formula V, of formula VI, or of formula VII, as hereinbefore defined, for example under conditions known to those skilled in the art (e.g. conditions described at process steps (b), (c) and (d) above in respect of compounds of formula I).

Compounds of formula X may be prepared by oxidation of a corresponding compound of formula XV,

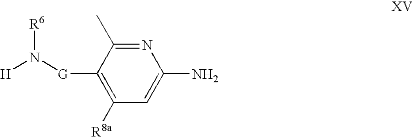

or an N-protected or N,N′-diprotected derivative thereof, wherein R⁶ and R^{8a} are as hereinbefore defined, in the presence of a suitable oxidising agent (e.g. mCPBA), for example under conditions known to those skilled in the art (e.g. at sub-ambient temperature (such as 0° C.) in the presence of a suitable solvent (such as DCM)).

Suitable protected derivatives of compounds of formulae X and XV for use in the preparation of compounds of formula III include the N,N′-di(tert-butyloxycarbonyl)-protected (di-Boc-protected) compounds.

Compounds of formula XII may be prepared by nitrosation of a corresponding compound of formula XVI,

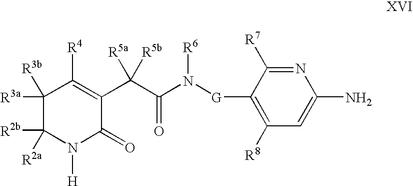

or a derivative thereof that is protected at the 2-amino substituent of the pyridine ring, wherein R^{2a}, R^{2b}, R^{3a}, R^{3b}, R⁴, R^{5a}, R^{5b}, R⁶ to R⁸ and G are as hereinbefore defined, for example under conditions well known to those skilled in the art, e.g. reaction at with a nitrosating agent (such as nitrous acid, NOCl, N₂O₃, N₂O₄ or, particularly, a C₁₋₆ alkyl nitrite (e.g. tert-butyl nitrite)) in the presence of a suitable solvent (e.g. diethyl ether) and optionally in the presence of an appropriate base (e.g. pyridine).

Compounds of formula XIII may be prepared by reduction of a carboxylic acid of formula XVII,

R¹—C₀₋₅ alkylene-C(O)OH XVII

wherein R¹ is as hereinbefore defined, for example under conditions known to those skilled in the art, such as reaction with LiAlH₄ or, particularly, borane in the presence of a suitable solvent (such as THF).

Compounds of formula XIV may be prepared by reduction of a compound of formula XVIII,

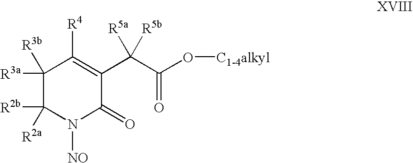

wherein R^{2a}, R^{2b}, R^{3a}, R^{3b}, R⁴, R^{5a} and R^{5b} are as hereinbefore defined, for example under conditions described hereinbefore in respect of the preparation of compounds of formula IV.

Compounds of formula XIV may alternatively be prepared by reaction of a compound of formula XIX,

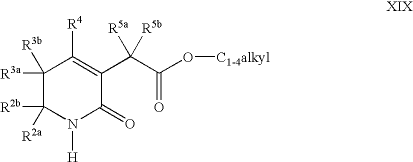

wherein R^{2a}, R^{2b}, R^{3a}, R^{3b}, R⁴, R^{5a} and R^{5b} are as hereinbefore defined, with O-(diphenylphosphinyl)hydroxylamine or O-(2,4-dinitrophenyl)-hydroxylamine, for example under conditions known to those skilled in the art (e.g. at ambient temperature (such as 15 to 25° C.) in the presence of an appropriate base (such as Cs₂CO₃ or NaH) and a suitable solvent (such as DMF)).

Compounds of formula XVI may be prepared by analogy with compounds of formulae I and XIX.

Compounds of formula XVIII may be prepared by nitrosation of a corresponding compound of formula XIX, as hereinbefore defined, for example under conditions described hereinbefore in respect of the preparation of compounds of formula XII.

Compounds of formula XIX may be prepared by α,β-elimination (relative to the oxo group of the piperidinone ring) of H-Lg³ from a piperidinone of formula XX,

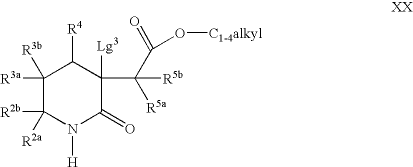

or a protected derivative thereof, wherein Lg³ represents a leaving group capable of undergoing thermal 1,2-elimination (e.g. —Se(O)-phenyl) and R^{2a}, R^{2b}, R^{3a}, R^{3b}, R⁴, R^{5a} and R^{5b} are as hereinbefore defined, for example under conditions that are well known to those skilled in the art (e.g. when Lg³ represents —Se(O)-phenyl, thermal elimination of Ph-Se—OH at ambient temperature (such as 15 to 25° C.) in the presence of a suitable solvent (such as DCM, water or a mixture thereof)).

Compounds of formula XX in which Lg³ represents —Se(O)-phenyl may be prepared by oxidation of a compound of formula XXI,

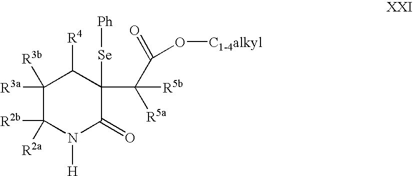

or a protected derivative thereof, wherein R^{2a}, R^{2b}, R^{3a}, R^{3b}, R⁴, R^{5a} and R^{5b} are as hereinbefore defined, for example under conditions well know to those skilled in the art (e.g. reaction at sub-ambient temperature (such as 0° C.) with an appropriate oxidising agent (such as mCPBA or, particularly, hydrogen peroxide) in the presence of a suitable solvent (such as DCM, water or a mixture thereof)).

As the skilled person will appreciate, the conversion of compounds of formula XXI to corresponding compounds of formula XIX may conveniently take place in a “one-pot” procedure, where the oxidised intermediate (the compound of formula XX in which Lg³ represents —Se(O)-phenyl) is not isolated and thermal elimination of Ph-Se—OH takes place during the “work-up” of the oxidation reaction.

Compounds of formula XXI may be prepared by reaction of a compound of formula XXII,

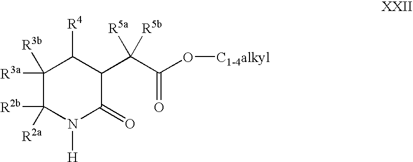

or a protected derivative thereof, wherein R^{2a}, R^{2b}, R^{3a}, R^{3b}, R⁴, R^{5a} and R^{5b} are as hereinbefore defined, with a compound of formula XXIII,

Phenyl-Se-Lg⁴ XXIII

wherein Lg⁴ represents a suitable leaving group (e.g. halo, such as Br, or —SePh), in the presence of an appropriate base (e.g. a metal hydride or, particularly, a metal amide (such as lithium bis(trimethylsilyl)amide)), for example under conditions known to those skilled in the art (e.g. at low temperature (such as −78° C.)) in the presence of a suitable solvent (such as THF).

Compounds of formula XXII may be prepared by reaction of a compound of formula XXIV,

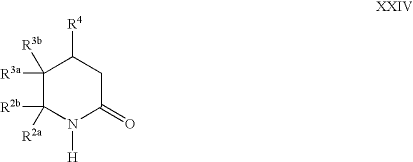

or a protected derivative thereof, wherein R^{2a}, R^{2b}, R^{3a}, R^{3b} and R⁴ are as hereinbefore defined, with a compound of formula XXV,

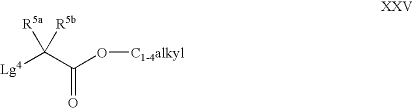

wherein Lg⁴, R^{5a} and R^{5b} are as hereinbefore defined, in the presence of an appropriate base (e.g. a metal hydride or, particularly, a metal amide (such as lithium bis(trimethylsilyl)amide)), for example under conditions known to those skilled in the art (e.g. at low temperature (such as −78 to −10° C.)) in the presence of a suitable solvent (such as THF).

Compounds of formula XXIV may be prepared by oxidation of a compound of formula XXVI,

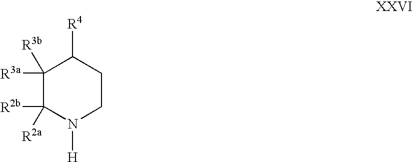

or a protected derivative thereof, wherein R^{2a}, R^{2b}, R^{3a}, R^{3b} and R⁴ are as hereinbefore defined, with a suitable oxidising agent (e.g. H₂O₂, (PhIO)ₙ, Hg(OAc)₂ or, particularly, RuO₄, which latter reagent may be formed in situ by oxidation of RuO₂ (e.g. by an excess of NaIO₄)), for example under conditions known to those skilled in the art (e.g. at ambient temperature (such as 15 to 25° C.) in the presence of a suitable solvent (such as ethyl acetate, water or a mixture thereof)).

As the skilled person will appreciate, the conversion of compounds of formula XXVI to corresponding compounds of formula XIX may require, at any or all of the reaction steps, protection of the NH group of the piperidone ring system. Suitable protective groups for this purpose include benzyloxycarbonyl and, particularly, tert-butyloxycarbonyl. The protective group may be introduced and removed under conditions that are well known to those skilled in the art. The protective group may be conveniently introduced before the compound of formula XXVI is converted to the compound of XXIV (e.g. by reaction, under conditions that are well known to those skilled in the art, of a compound of XXVI with di-tert-butyldicarbonate). Further, the protective group may be conveniently removed, again under conditions that are well known to those skilled in the art (e.g. by reaction with trifluoroacetic acid), once the compound of formula XIX has been formed.

Compounds of formulae V, VI, VIII, XI, XV, XVII, XXIII, XXV and XXVI are either commercially available, are known in the literature, or may be obtained by analogy with the processes described herein, or by conventional synthetic procedures, in accordance with standard techniques, from readily available starting materials using appropriate reagents and reaction conditions. In this respect, compounds described herein may also be obtained by analogy with synthetic procedures described in the prior art documents mentioned above (and WO 94/20467, WO 94/29336, WO 95/23609, WO 96/06832, WO 96/06849, WO 97/11693, WO 97/24135, WO 98/01422, WO 01/68605, WO 99/26920, WO 01/79155, WO 01/68605, WO 96/18644, WO 97/01338, WO 97/30708, WO 98/16547, WO 99/26926, WO 00/73302, WO 01/04117, WO 01/79262, WO 02/057225, WO 02/064140, WO 03/29224, U.S. Pat. No. 5,668,289, U.S. Pat. No. 5,792,779 and WO 95/35313 in particular).

Substituents on alkyl, alkenyl, cycloalkyl, cycloalkenyl, aryl and heterocyclic groups in compounds of formulae I to XXII and XXIII to XXVI may be introduced and/or interconverted using techniques well known to those skilled in the art by way of standard functional groups interconversions, in accordance with standard techniques, from readily available starting materials using appropriate reagents and reaction conditions. For example, hydroxy may be esterified or converted to alkoxy, acyloxy may be hydrolysed to hydroxy, phenyl may be halogenated to give halophenyl, halo may be displaced by cyano, etc.

The skilled person will also appreciate that various standard substituent or functional group interconversions and transformations within certain compounds of formula I will provide other compounds of formula I. For example, hydroxy may be esterified to provide acetyloxy or benzoyloxy.

Compounds of formula I may be isolated from their reaction mixtures using conventional techniques.

In accordance with the present invention, pharmaceutically acceptable derivatives of compounds of formula I also include “protected” derivatives, and/or compounds that act as prodrugs, of compounds of formula I.

Protected derivatives of compounds of formula I that may be mentioned include derivatives in which the amino (NH₂) substituent on the 2,4-dialkyl-6-aminopyridin-3-yl group bears an amino protective group (such as ter t-butyloxycarbonyl, benzyloxycarbonyl and the like). Such protective groups may also be utilised in the synthesis of compounds of formula I (e.g. they may be present on the 2-amino substituent of the pyridinyl group in protected derivatives of compounds of formulae III and IV).

Compounds that may act as prodrugs of certain compounds of formula I (e.g. compounds of formula I in which R⁷ and/or R⁸ is substituted by OH) that may be mentioned include compounds of formula I in which R⁷ and/or R⁸ is substituted by O—C(O)—X—R¹¹.

The compounds of the invention may exhibit tautomerism. All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography. The various stereoisomers may be isolated by separation of a racemic or other mixture of the compounds using conventional, e.g. HPLC techniques. Alternatively the desired optical isomers may be made by reaction of the appropriate optically active starting materials under conditions which will not cause racemisation or epimerisation, or by derivatisation, for example with a homochiral acid followed by separation of the diastereomeric derivatives by conventional means (e.g. HPLC, chromatography over silica). All stereoisomers are included within the scope of the invention.

It will be appreciated by those skilled in the art that in the processes described above and hereinafter the functional groups of intermediate compounds may need to be protected by protecting groups.

Functional groups that it is desirable to protect include hydroxy, amino and carboxylic acid. Suitable protecting groups for hydroxy include optionally substituted and/or unsaturated alkyl groups (e.g. methyl, allyl, benzyl or tert-butyl), trialkylsilyl or diarylalkylsilyl groups (e.g. t-butyldimethylsilyl, t-butyldiphenylsilyl or trimethylsilyl) and tetrahydropyranyl. Suitable protecting groups for carboxylic acid include C₁₋₆ alkyl or benzyl esters. Suitable protecting groups for amino and amidino include t-butyloxycarbonyl, benzyloxycarbonyl or 2-trimethylsilylethoxycarbonyl (Teoc). Amidino nitrogens may also be protected by hydroxy or alkoxy groups, and may be either mono- or diprotected.

The protection and deprotection of functional groups may take place before or after coupling, or before or after any other reaction in the above-mentioned schemes.

Protecting groups may be removed in accordance with techniques that are well known to those skilled in the art and as described hereinafter.

Persons skilled in the art will appreciate that, in order to obtain compounds of the invention in an alternative, and, on some occasions, more convenient, manner, the individual process steps mentioned hereinbefore may be performed in a different order, and/or the individual reactions may be performed at a different stage in the overall route (i.e. substituents may be added to and/or chemical transformations performed upon, different intermediates to those mentioned hereinbefore in conjunction with a particular reaction). This may negate, or render necessary, the need for protecting groups.

The type of chemistry involved will dictate the need, and type, of protecting groups as well as the sequence for accomplishing the synthesis.

The use of protecting groups is described in “Protective Groups in Organic Chemistry”, edited by J W F McOmie, Plenum Press (1973), and “Protective Groups in Organic Synthesis”, 3rd edition, T. W. Greene & P. G. M. Wutz, Wiles Interscience (1999).

Protected derivatives of compounds of the invention may be converted chemically to compounds of the invention using standard deprotection techniques (e.g. hydrogenation). The skilled person will also appreciate that certain compounds of formula I (e.g. compounds in which R⁷ and/or R⁸ is substituted by O—C(O)—X—R¹¹) may also be referred to as being “protected derivatives” of other compounds of formula I (e.g. those in which R⁷ and/or R⁸ is substituted by OH).

Those skilled in the art will also appreciate that certain compounds of formula I will be useful as intermediates in the synthesis of certain other compounds of formula I.

Some of the intermediates referred to hereinbefore are novel. According to a further aspect of the invention there is thus provided: (a) a compound of formula III, or a protected derivative thereof; (b) a compound of formula IV, or a protected derivative thereof; (c) a compound of formula X, or a protected derivative thereof; (d) a compound of formula XII, or a protected derivative thereof; and (e) a compound of formula XVI, or a protected derivative thereof.

### Medical and Pharmaceutical Use

Compounds of the invention may possess pharmacological activity as such. However, other compounds of the invention (including compounds of formula I in which R⁷ and/or R⁸ is substituted by O—C(O)—X—R¹¹) may not possess such activity, but may be administered parenterally or orally, and may thereafter be metabolised in the body to form compounds that are pharmacologically active (including, but not limited to, corresponding compounds of formula I in which R⁷ and/or R⁸ is substituted by OH). Such compounds (which also includes compounds that may possess some pharmacological activity, but that activity is appreciably lower than that of the “active” compounds to which they are metabolised), may therefore be described as “prodrugs” of the active compounds.

Thus, the compounds of the invention are useful because they possess pharmacological activity, and/or are metabolised in the body following oral or parenteral administration to form compounds which possess pharmacological activity. The compounds of the invention are therefore indicated as pharmaceuticals.

According to a further aspect of the invention there is thus provided the compounds of the invention for use as pharmaceuticals.

In particular, compounds of the invention are potent inhibitors of thrombin either as such and/or (e.g. in the case of prodrugs), are metabolised following administration to form potent inhibitors of thrombin, for example as may be demonstrated in the tests described below.

By “prodrug of a thrombin inhibitor”, we include compounds that form a thrombin inhibitor, in an experimentally-detectable amount, and within a predetermined time (e.g. about 1 hour), following oral or parenteral administration (see, for example, Test E below) or, alternatively, following incubation in the presence of liver microsomes (see, for example, Test F below).

The compounds of the invention are thus expected to be useful in those conditions where inhibition of thrombin is beneficial (as determined by reference to a clinically relevant end-point, e.g. conditions, such as thrombo-embolisms, where inhibition of thrombin is required or desired, and/or conditions where anticoagulant therapy is indicated), including the following:

The treatment and/or prophylaxis of thrombosis and hypercoagulability in blood and/or tissues of animals including man. It is known that hypercoagulability may lead to thrombo-embolic diseases. Conditions associated with hypercoagulability and thrombo-embolic diseases are usually designated as thrombophilia conditions. These conditions include, but are not limited to, inherited or acquired activated protein C resistance, such as the factor V-mutation (factor V Leiden), inherited or acquired deficiencies in antithrombin III, protein C, protein S, heparin cofactor II, and conditions with increased plasma levels of the coagulation factors such as caused by the prothrombin G20210A mutation. Other conditions known to be associated with hypercoagulability and thrombo-embolic disease include circulating antiphospholipid antibodies (Lupus anticoagulant), homocysteinemi, heparin induced thrombocytopenia and defects in fibrinolysis, as well as coagulation syndromes (e.g. disseminated intravascular coagulation (DIC)) and vascular injury in general (e.g. due to trauma or surgery). Furthermore, low physical activity, low cardiac output or high age are known to increase the risk of thrombosis and hypercoagulability may be just one of several factors underlying the increased risk. These conditions include, but are not limited to, prolonged bed rest, prolonged air travelling, hospitalisation for an acute medical disorder such as cardiac insufficiency or respiratory insufficiency. Further conditions with increased risk of thrombosis with hypercoagulability as on component are pregnancy and hormone treatment (e.g. oestrogen).

The treatment of conditions where there is an undesirable excess of thrombin without signs of hypercoagulability, for example in neurodegenerative diseases such as Alzheimer's disease.

Particular disease states which may be mentioned include the therapeutic and/or prophylactic treatment of venous thrombosis (e.g. deep venous thrombosis, DVT) and pulmonary embolism, arterial thrombosis (e.g. in myocardial infarction, unstable angina, thrombosis-based stroke and peripheral arterial thrombosis), and systemic embolism usually from the atrium during atrial fibrillation (e.g. non valvular or valvular atrial fibrillation) or from the left ventricle after transmural myocardial infarction, or caused by congestive heart failure; prophylaxis of re-occlusion (i.e. thrombosis) after thrombolysis, percutaneous trans-luminal angioplasty (PTA) and coronary bypass operations; the prevention of thrombosis after microsurgery and vascular surgery in general.

Further indications include the therapeutic and/or prophylactic treatment of disseminated intravascular coagulation caused by bacteria, multiple trauma, intoxication or any other mechanism; anticoagulant treatment when blood is in contact with foreign surfaces in the body such as vascular grafts, vascular stents, vascular catheters, mechanical and biological prosthetic valves or any other medical device; and anticoagulant treatment when blood is in contact with medical devices outside the body such as during cardiovascular surgery using a heart-lung machine or in haemodialysis; the therapeutic and/or prophylactic treatment of idiopathic and adult respiratory distress syndrome, pulmonary fibrosis following treatment with radiation or chemotherapy, chronic obstructive lung disease, septic shock, septicemia, inflammatory responses, which include, but are not limited to, edema, acute or chronic atherosclerosis such as coronary arterial disease and the formation of atherosclerotic plaques, cardiac insufficiency, cerebral arterial disease, cerebral infarction, cerebral thrombosis, cerebral embolism, peripheral arterial disease, ischaemia, angina (including unstable angina), reperfusion damage, restenosis after percutaneous trans-luminal angioplasty (PTA) and coronary artery bypass surgery.

Compounds of the invention that inhibit trypsin and/or thrombin may also be useful in the treatment of pancreatitis.

The compounds of the invention are thus indicated both in the therapeutic and/or prophylactic treatment of these conditions.

According to a further aspect of the present invention, there is provided a method of treatment of a condition where inhibition of thrombin is required which method comprises administration of a therapeutically effective amount of a compound of the invention to a person suffering from, or susceptible to, such a condition.

The compounds of the invention will normally be administered orally, intravenously, subcutaneously, buccally, rectally, dermally, nasally, tracheally, bronchially, by any other parenteral route or via inhalation, in the form of pharmaceutical preparations comprising compound of the invention either as a free base, or a pharmaceutically acceptable nontoxic organic or inorganic acid addition salt, in a pharmaceutically acceptable dosage form.

Preferred route of administration of compounds of the invention are oral.

Depending upon the disorder and patient to be treated and the route of administration, the compositions may be administered at varying doses.

The compounds of the invention may also be combined and/or co-administered with any antithrombotic agent(s) with a different mechanism of action, such as one or more of the following: the anticoagulants unfractionated heparin, low molecular weight heparin, other heparin derivatives, synthetic heparin derivatives (e.g. fondaparinux), vitamin K antagonists, synthetic or biotechnological inhibitors of other coagulation factors than thrombin (e.g. synthetic FXa, FVIIa and FIXa inhibitors, and rNAPc2), the antiplatelet agents acetylsalicylic acid, ticlopidine and clopidogrel; thromboxane receptor and/or synthetase inhibitors; fibrinogen receptor antagonists; prostacyclin mimetics; phosphodiesterase inhibitors; ADP-receptor (P2X₁, P2Y₁, P2Y₁₂ [P₂T]) antagonists; and inhibitors of carboxypeptidase U (CPU or TAFIa) and inhibitors of plasminogen activator inhibitor-1 (PAI-1).

The compounds of the invention may further be combined and/or co-administered with thrombolytics such as one or more of tissue plasminogen activator (natural, recombinant or modified), streptokinase, urokinase, prourokinase, anisoylated plasminogenstreptokinase activator complex (APSAC), animal salivary gland plasminogen activators, and the like, in the treatment of thrombotic diseases, in particular myocardial infarction.

According to a further aspect of the invention there is thus provided a pharmaceutical formulation including a compound of the invention, in admixture with a pharmaceutically acceptable adjuvant, diluent or carrier.

Suitable daily doses of the compounds of the invention in therapeutic treatment of humans are about 0.001-100 mg/kg body weight at peroral administration and 0.001-50 mg/kg body weight at parenteral administration.

For the avoidance of doubt, as used herein, the term “treatment” includes therapeutic and/or prophylactic treatment.

Compounds of the invention have the advantage that they may be more efficacious, be less toxic, be longer acting, have a broader range of activity, be more selective (e.g. for inhibiting thrombin over other serine proteases, in particular trypsin and those involved in haemostasis), be more potent, produce fewer side effects, be more easily absorbed, and/or have a better pharmacokinetic profile (e.g. higher oral bioavailability and/or lower clearance), than, and/or have other useful pharmacological, physical, or chemical, properties over, compounds known in the prior art.

### Biological Tests

The following test procedures may be employed.

### Test A

### Determination of Thrombin Clotting Time (TT)

The inhibitor solution (25 μL) is incubated with plasma (25 μL) for three minutes. Human thrombin (T 6769; Sigma Chem. Co or Hematologic Technologies) in buffer solution, pH 7.4 (25 μL, 4.0 NIH units/1 mL), is then added and the clotting time measured in an automatic device (KC 10; Amelung).

The thrombin clotting time (TT) is expressed as absolute values (seconds) as well as the ratio of TT without inhibitor (TT₀) to TT with inhibitor (TTᵢ). The latter ratios (range 1-0) are plotted against the concentration of inhibitor (log transformed) and fitted to sigmoidal dose-response curves according to the equation

y=a/[1+(x/IC₅₀)^{s}]

where: a=maximum range, i.e. 1; s=slope of the dose-response curve; and IC₅₀=the concentration of inhibitor that doubles the clotting time. The calculations are processed on a PC using the software program GraFit Version 3, setting equation equal to: Start at 0, define end=1 (Erithacus Software, Robin Leatherbarrow, Imperial College of Science, London, UK).

### Test B

Determination of Thrombin Inhibition with a Chromogenic, Robotic Assay

The thrombin inhibitor potency is measured with a chromogenic substrate method, in a Plato 3300 robotic microplate processor (Rosys AG, CH-8634 Hombrechtikon, Switzerland), using 96-well, half volume microtitre plates (Costar, Cambridge, Mass., USA; Cat No 3690). Stock solutions of test substance in DMSO (72 μL), 0.1-1 mmol/L, are diluted serially 1:3 (24+48 μL) with DMSO to obtain ten different concentrations, which are analysed as samples in the assay. 2 μL of test sample is diluted with 124 μL assay buffer, 12 μL of chromogenic substrate solution (S-2366, Chromogenix, Mölndal, Sweden) in assay buffer and finally 12 ILL of a-thrombin solution (Human a-thrombin, Sigma Chemical Co. or Hematologic Technologies) in assay buffer, are added, and the samples mixed. The final assay concentrations are: test substance 0.00068-133 μmol/L, S-2366 0.30 mmol/L, a-thrombin 0.020 NIHU/mL. The linear absorbance increment during 40 minutes incubation at 37° C. is used for calculation of percentage inhibition for the test samples, as compared to blanks without inhibitor. The IC₅₀-robotic value, corresponding to the inhibitor concentration which causes 50% inhibition of the thrombin activity, is calculated from a log concentration vs. % inhibition curve.

### Test C

### Determination of the Inhibition Constant Kᵢ for Human Thrombin

Kᵢ-determinations are made using a chromogenic substrate method, performed at 37° C. on a Cobas Bio centrifugal analyser (Roche, Basel, Switzerland). Residual enzyme activity after incubation of human I-thrombin with various concentrations of test compound is determined at three different substrate concentrations, and is measured as the change in optical absorbance at 405 nm.

Test compound solutions (100 μL; normally in buffer or saline containing BSA 10 g/L) are mixed with 200 μL of human a-thrombin (Sigma Chemical Co) in assay buffer (0.05 mol/L Tris-HCl pH 7.4, ionic strength 0.15 adjusted with NaCl) containing BSA (10 g/L), and analysed as samples in the Cobas Bio. A 60 μL sample, together with 20 μL of water, is added to 320 mL of the substrate S-2238 (Chromogenix AB, Mölndal, Sweden) in assay buffer, and the absorbance change (?A/min) is monitored. The final concentrations of S-2238 are 16, 24 and 50 μmol/L and of thrombin 0.125 NIH U/mL.

The steady state reaction rate is used to construct Dixon plots, i.e. diagrams of inhibitor concentration vs. 1/(?A/min). For reversible, competitive inhibitors, the data points for the different substrate concentrations typically form straight lines which intercept at x=−Kᵢ.

### Test D

### Determination of Activated Partial Thromboplastin Time (APTT)

APTT is determined in pooled normal human citrated plasma with the reagent PTT Automated 5 manufactured by Stago. The inhibitors are added to the plasma (10 μL inhibitor solution to 90 μL plasma) and incubated with the APTT reagent for 3 minutes followed by the addition of 100 μL of calcium chloride solution (0.025 M) and APTT is determined by use of the coagulation analyser KC10 (Amelung) according to the instructions of the reagent producer.

The clotting time is expressed as absolute values (seconds) as well as the ratio of APTT without inhibitor (APTTO) to APTT with inhibitor (APTTI). The latter ratios (range 1-0) are plotted against the concentration of inhibitor (log transformed) and fitted to sigmoidal dose-response curves according to the equation

y=a/[1+(x/IC₅₀)^{s}]

where: a=maximum range, i.e. 1; s=slope of the dose-response curve; and IC₅₀=the concentration of inhibitor that doubles the clotting time. The calculations are processed on a PC using the software program GraFit Version 3, setting equation equal to: Start at 0, define end=1 (Erithacus Software, Robin Leatherbarrow, Imperial College of Science, London, UK).

IC₅₀APTT is defined as the concentration of inhibitor in human plasma that doubled the Activated Partial Thromboplastin Time.

### Test E

### Determination of Plasma Clearance and Oral Bioavailability in Rat

Plasma clearance and oral bioavailability are estimated in female Sprague Dawley rats. The compound is dissolved in water or another appropriate vehicle. For determination of plasma clearance the compound is administered as a subcutaneous (sc) or an intravenous (iv) bolus injection at a dose of 1-4 μmol/kg. Blood samples are collected at frequent intervals up to 24 hours after drug administration. For bioavailability estimates, the compound is administered orally at 10 μmol/kg via gavage and blood samples are collected frequently up to 24 hours after dosing. The blood samples are collected in heparinized tubes and centrifuged within 30 minutes, in order to separate the plasma from the blood cells. The plasma is transferred to plastic vials with screw caps and stored at −20° C. until analysis. Prior to the analysis, the plasma is thawed and 50 μL of plasma samples are precipitated with 150 μL of cold acetonitrile. The samples are centrifuged for 20 minutes at 4000 rpm. 75 μL of the supernatant is diluted with 75 μL of 0.2% formic acid. 10 μL volumes of the resulting solutions are analysed by LC-MS/MS and the concentrations of thrombin inhibitor are determined using standard curves. All pharmacokinetic calculations are performed with the computer program WinNonlinTMProfessional (Pharsight Corporation, California, USA), or an equivalent program. Area under the plasma concentration-time profiles (AUC) is estimated using the log/linear trapezoidal rule and extrapolated to infinite time. Plasma clearance (CL) of the compound is then determined as

CL=Dose(iv/sc)/AUC(iv/sc).

The oral bioavailability is calculated as

F=CL×AUC(po)/Dose(po).

Plasma clearance is reported as mL/min/kg and oral bioavailability as percentage (%).

### Test F

### Determination of In Vitro (Liver Microsome) Stability

Liver microsomes are prepared from Sprague-Dawley rats and human liver samples according to internal SOPs. The compounds are incubated at 37° C. at a total microsome protein concentration of 0.5 mg/mL in a 0.1 mol/L potassium phosphate buffer at pH 7.4, in the presence of the cofactor, NADPH (1.0 mmol/L). The initial concentration of compound is 1.0 μmol/L. Samples are taken for analysis at 5 time points, 0, 7, 15, 20 and 30 minutes after the start of the incubation. The enzymatic activity in the collected sample is immediately stopped by adding an equal volume of acetonitrile containing 0.8% formic acid. The concentration of compound remaining in each of the collected samples is determined by means of LC-MS/MS. The elimination rate constant (k) of the thrombin inhibitor is calculated as the slope of the plot of ln[Thrombin inhibitor] against incubation time (minutes). The elimination rate constant is then used to calculate the half-life (T_{1/2}) of the thrombin inhibitor, which is subsequently used to calculate the intrinsic clearance (CLint) of the thrombin inhibitor in liver microsomes as:

$$CLint ( in μ L \text{/} min \text{/} mg ) = \frac{( ln 2 \times incubation volume )}{( {T}_{1 / 2} \times protein concentration )}$$

### Test G

### Venous Thrombosis Model

The thrombogenic stimuli are vessel damage and blood flow stasis. Rats are anaesthetised and the abdomen is opened. A partial occlusion on the caval vein, caudal to the left kidney-vein, is obtained with a snare around the vein and a cannula, which is than removed. A filter-paper soaked with FeCl₃ is placed on the external surface of the distal part of the caval vein. The abdomen is filled with saline and closed. At the end of the experiment the rat is sacrificed, the caval vein is extirpated, the thrombus harvested and its wet weight determined.

### EXAMPLES

### General Experimental Procedures

High resolution mass spectra were recorded on a Micromass LCT mass spectrometer equipped with an electrospray interface (LC-HRMS). ¹H NMR measurements were performed on Varian UNITY plus 400, 500 and 600 spectrometers, operating at ¹H frequencies of 400, 500 and 600 MHz respectively. Chemical shifts are given in ppm with the solvent as internal standard. Flash chromatography separations were performed using Merck Silica gel 60 (0.063-0.200 mm). The compounds named below were named using ACD/name version 8.05/13 Apr. 2004 available from Advanced Chemistry Development Inc., Canada.

### Preparation of Intermediates

### Preparation 1

### (1-Amino-4-methyl-2-oxo-1,2,5,6-tetrahydropyridin-3-yl)acetic acid ethyl ester

### (a) 4-Methylpiperidine-1-carboxylic acid tert-butyl ester

4-Methylpiperidine (5.0 g, 50 mmol) and di-tert-butyl dicarbonate (13 g, 60 mmol) were dissolved in DCM (50 mL). TEA (7.65 mL, 1.1 mol equiv.) was added and the reaction mixture was stirred at 35° C. for 3 hours. The solvent was removed in vacuo and the residue was purified by flash chromatography (SiO₂, hexane) to give the sub-title compound (7.29 g, 73%).

¹H NMR (400 MHz, CDCl₃) δ 0.81 (d, 3H), 0.86-1.00 (m, 2H), 1.33 (s, 9H), 1.13-1.49 (m, 3H), 2.55 (m, 2H), 3.93 (m, 2H)

### (b) 4-Methyl-2-oxopiperidine-1-carboxylic acid tert-butyl ester

4-Methyl-piperidine-1-carboxylic acid tert-butyl ester (1.1 g, 5.5 mmol; see step (a) above) was dissolved in ethyl acetate (70 mL) and was added to a solution of ruthenium oxide (0.020 g, 0.15 mmol) and sodium periodate (4.5 g, 21 mmol) dissolved in water (215 mL). The reaction was stirred vigorously under air for 18 hours. The layers were separated and the aqueous phase was extracted with ethyl acetate. The combined organic extracts were dried and filtered through Celite®. The solvent was removed in vacuo and the residue (the sub-title compound—0.98 g, 83%) was used without further purification.

¹H NMR (400 MHz, CDCl₃) δ 1.02 (d, 3H), 1.43-1.57 (m, 1H), 1.53 (s, 9H), 1.90-2.03 (m, 2H), 2.04-2.30 (m, 1H), 2.56-2.62 (m, 1H), 3.46-3.53 (m, 1H), 3.78-3.82 (m, 1H)

### (c) 3-Ethoxycarbonylmethyl-4-methyl-2-oxo-piperidine-1-carboxylic acid tert-butyl ester

Lithium bis(trimethylsilyl)amide (2.1 mL, 1 M in THF, 2.1 mmol) was added slowly to a solution of 4-methyl-2-oxopiperidine-1-carboxylic acid tert-butyl ester (0.40 g, 1.87 mmol; see step (b) above) in THF (7 mL) at −78° C. The solution was stirred for 40 minutes. Ethyl bromoacetate (0.31 mL, 2.8 mmol, 1.5 mol equiv.) was added at −78° C. and the reaction mixture was warmed to −20° C. over a period of 2 hours. The reaction was quenched by addition of ammonium chloride (sat., 10 mL). The mixture was diluted with ethyl acetate (30 mL) and the layers were separated. The aqueous phase was extracted with ethyl acetate (3×25 mL). The combined organic layers were dried (Na₂SO₄), filtered and concentrated under reduced pressure. Purification by flash chromatography (SiO₂, 10-20% ethyl acetate in hexane) gave the sub-title compound (0.387 g, 69%) as a colourless oil.

¹H NMR (400 MHz, CDCl₃) δ 0.95 (d, 3H) 1.15 (t, 3H), 1.33-1.47 (m, 1H), 1.41 (s, 9H), 1.79-1.93 (m, 2H), 2.29-2.34 (m, 1H), 2.59 (dd, 1H), 2.69 (dd, 1H), 3.51-3.56 (m, 1H), 3.57-3.67 (m, 1H), 4.03 (q, 2H)

### (d) 5-Ethoxycarbonylmethyl-4-methyl-6-oxo-3,6-dihydro-2H-pyridine-1-carboxylic acid tert-butyl ester

Lithium bis(trimethylsilyl)amide (3.1 mL, 1 M in THF, 3.1 mmol) was added slowly to a solution of 3-ethoxycarbonylmethyl-4-methyl-2-oxo-piperidine-1-carboxylic acid tert-butyl ester (0.77 g, 2.6 mmol; see step (c) above) in THF (26 mL) at −78° C. The solution was stirred for 90 minutes and then phenylselenium bromide (0.80 g, 3.4 mmol) in THF (2×3 mL) was added at −78° C. The reaction mixture was stirred at −78° C. for 90 minutes and was then warmed to −20° C. over a period of 2 hours and quenched by addition of ammonium chloride (sat., 60 mL). The mixture was diluted with ethyl acetate (50 mL) and the layers were separated. The aqueous phase was extracted with ethyl acetate (3×25 mL). The combined organic layers were dried (Na₂SO₄), filtered and concentrated under reduced pressure.

The residue was dissolved in DCM (10 mL) and cooled to 0° C. Hydrogen peroxide (30%, 10 mL) was added and the pH was adjusted to 7 with pyridine. The reaction mixture was allowed to warm to room temperature. The reaction mixture was quenched after 10 minutes at 0° C. with ammonium chloride (sat., 60 mL) and the mixture was extracted with DCM (50 mL). The organic phase was washed with brine, dried and the solvent was removed in vacuo. Purification and separation by flash chromatography (SiO₂, 20-60% ethyl acetate/hexane) gave the endocyclic compound (the sub-title compound—0.387 g, 69%) and the exocyclic compound as colourless oils.

The endocyclic compound was used in the next step.

Endocyclic compound:

¹H NMR (400 MHz, CDCl₃) δ 1.24 (t, 3H), 1.52 (s, 9H), 1.93 (s, 3H), 2.41 (t, 2H), 3.40 (br s, 2H), 3.81 (t, 2H), 4.12 (q, 2H)

### (e) (4-Methyl-2-oxo-1,2,5,6-tetrahydropyridin-3-yl)acetic acid ethyl ester

TFA (0.1 mL, 0.1 volume equiv.) was added to a solution of 5-ethoxy-carbonylmethyl-4-methyl-6-oxo-3,6-dihydro-2H-pyridine-1-carboxylic acid tert-butyl ester (0.025 g, 0.084 mmol; see step (d) above) in DCM (1 mL) and the reaction was stirred for 4 hours at room temperature. The TFA was removed under reduced pressure azeotropically with benzene (3×20 mL) to give the sub-title compound (deprotected amine), which was used in the next step without further purification.

### (f) (4-Methyl-1-nitroso-2-oxo-1,2,5,6-tetrahydropyridin-3-yl)acetic acid ethyl ester

The sub-title compound was prepared from the compound of step (e) above by one of the following two methods.

### Method A

tert-Butyl nitrite (0.015 mL, 0.13 mmol, 1.5 mol equiv.) and pyridine (0.020 mL, 0.25 mmol, 3 mol equiv.) were added to the solution of the crude amine (from step (e) above) in dry diethyl ether (1 mL). The reaction mixture was heated to reflux for 16 hours. An additional aliquot of tert-butyl nitrite (0.010 mL, 0.084 mmol, 1 mol equiv.) was added and reflux was continued for 16 hours. The solvent was removed under reduced pressure and purification by flash chromatography (SiO₂, 50% ethyl acetate in hexane) gave the sub-title compound (0.0174 g, 91%) as a yellow oil.

### Method B

The crude amine (738 mg, 3.74 mmol; from step (e) above) was dissolved in water (7 mL) and dimethoxyethane (3.5 mL). Hydrochloric acid (0.7 mL, conc.) was added and the mixture was cooled to 0° C. Sodium nitrite (309 mg, 4.49 mmol) dissolved in water (3.5 mL) was added in portions of 600 mL, and the reaction mixture was stirred whilst gradually warming to room temperature. After 2.5 hours, another portion of sodium nitrite (36 mg) in water (1 mL) was added and stirring was continued for 45 minutes. The reaction mixture was extracted with DCM and the organic phase was dried through a phase separator. The solvent was evaporated under reduced pressure and purification by flash chromatography (SiO₂, hexane:ethyl acetate 2.1) gave the sub-title compound (535 mg, 63%)

¹H NMR (400 MHz, CDCl₃) δ 1.30 (t, 3H), 2.08 (s, 3H), 2.57 (t, 2H), 3.59 (s, 2H), 3.89 (t, 2H), 4.20 (q, 2H)

### (g) (1-Amino-4-methyl-2-oxo-1,2,5,6-tetrahydropyridin-3-yl)acetic acid ethyl ester

Zinc powder (0.014 g, 0.21 mmol, 3 mol equiv.) was added to a solution of (4-methyl-1-nitroso-2-oxo-1,2,5,6-tetrahydropyridin-3-yl)acetic acid ethyl ester (0.016 g, 0.071 mmol; see step (f) above) in a mixture of methanol and acetic acid (2 mL, 1:1) at 0° C. The ice bath was removed and after approximately 5 to 10 minutes the yellow colour had disappeared. The reaction mixture was filtered through Celite® and the filter cake was washed with methanol (3×5 mL). The solvent was removed under reduced pressure and the excess acetic acid was removed azeotropically with benzene (3×5 mL) to give the title compound, which was used without further purification.

¹H NMR (500 MHz, CDCl₃): d 4.49 (broad s, 1.4H), 4.17 (q, 2H), 3.60 (t, 2H), 3.43 (s, 2H), 2.51 (t, 2H), 1.92 (s, 3H), 1.29 (t, 3H).

### Preparation 2

### Ethyl{1-[(2,2-difluoro-2-pyridin-2-ylethyl)amino]-4-methyl-2-oxo-1,2,5,6-tetra-hydropyridin-3-yl}acetate

2,2-Difluoro-2-pyridin-2-ylethyl trifluoromethanesulfonate (1.235 g, 4.24 mmol; prepared according to the method described in Organic Process & Development, 2004, 8 (2), 192-200), (1-amino-4-methyl-2-oxo-1,2,5,6-tetrahydropyridin-3-yl)acetic acid ethyl ester 1.50 g of 60% purity material, 4.24 mmol) and 2,6-di-tert-butyl-4-methylpyridine (1.306 g, 6.36 mmol) were dissolved in 1,2-dichloroethane (17 mL). The reaction mixture was heated in the microwave oven (at 120° C.) for 20 min, before being concentrated under reduced pressure. Purification using flash chromatography (heptane/EtOAc, 20-100% EtOAc) gave 0.858 g (57%) of the title compound.

### Preparation 3

### {1-[(2,2-Difluoro-2-pyridin-2-ylethyl)amino]-4-methyl-2-oxo-1,2,5,6-tetra-hydropyridin-3-yl}acetic acid

Ethyl{1-[(2,2-difluoro-2-pyridin-2-ylethyl)amino]-4-methyl-2-oxo-1,2,5,6-tetra-hydropyridin-3-yl}acetate (0.858 g, 2.43 mmol; see Preparation 2 above) was dissolved in 30 mL of a 4:1 mixture of THF and water. LiOH (3.6 mL of a 1 M solution in water, 3.6 mmol) was added and the solution was stirred at rt overnight. The reaction mixture was concentrated and a few millilitres of water were added. The water was acidified to ˜pH 4 with 2 M HCl and then extracted (4×) with diethylether/DCM (1:2). The organic phase was evaporated to give 0.696 g, (86%) of the title compound.

### Preparation 4

### tert-Butyl({6-[(tert-butoxycarbonyl)amino]-2,4-dimethyl-1-oxidopyridin-3-yl}-methyl)carbamate

A solution of MCPBA (2.71 g, 11.0 mmol) in DCM (15 mL) was prepared and cooled to 0° C. To this cooled solution was added slowly a solution of tert-butyl ({6-[(tert-butoxycarbonyl)amino]-2,4-dimethylpyridin-3-yl}methyl)carbamate (3.514 g, 10.0 mmol; obtainable as described in WO 97/01338) in DCM (15 mL). The reaction mixture was allowed to reach rt overnight and DCM was added for dilution. The solution was washed with NaHCO₃ (3×) and the organic phase was dried and evaporated to give 3.696 g (97%) of the title compound.

### Preparation 5

### (6-[(tert-Butoxycarbonyl)amino]-3-{[(tert-butoxycarbonyl)amino]methyl}-4-methylpyridin-2-yl)methyl acetate

tert-Butyl({6-[(tert-butoxycarbonyl)amino]-2,4-dimethyl-1-oxidopyridin-3-yl}-methyl)carbamate (3.676 g, 10.0 mmol; see Preparation 4 above) was dissolved in acetic anhydride (40 mL) and warmed to 70° C. for 3 h. The reaction mixture was concentrated under reduced pressure, redissolved in EtOH and then concentrated under reduced pressure again. The resulting di-acetylated intermediate was dissolved in dry MeCN (35 mL) and treated with N,N-diethylethylenediamine (1.904 mL, 13.55 mmol) and stirred at rt for 2 h. Evaporation at reduced pressure gave a semi-solid residue which was partitioned between diethylether and 10% KHSO₄. The organic phase was thoroughly washed with 10% KHSO₄ (3×), NaHCO₃ (2×) and brine (2×), dried and concentrated under reduced pressure. Purification using flash chromatography (heptane/EtOAc, 10-60% EtOAc) gave is 1.931 g (47%) of the title compound.

### Preparation 6

### [6-Amino-3-(aminomethyl)-4-methylpyridin-2-yl]methanol

### (a) tert-Butyl{[6-[(tert-butoxycarbonyl)amino]-2-(hydroxymethyl)-4-methyl-pyridin-3-yl]methyl}carbamate

An aqueous solution of K₂CO₃ (1M, 9 mL, 9 mmol) was added to a solution of (6-[(tert-butoxycarbonyl)amino]-3-{[(tert-butoxycarbonyl)amino]methyl}-4-methyl-pyridin-2-yl)methyl acetate (1.851 g, 4.52 mmol; see Preparation 5 above) in MeOH (30 mL) at rt. The reaction mixture was stirred for 1 h at rt, after which time the solvent was removed under reduced pressure and the residue was dissolved in DCM and washed with brine. The organic phase was separated using a phase separator and was then concentrated under reduced pressure to give 1.58 g (95%) of the sub-title compound.

### (b) [6-Amino-3-(aminomethyl)-4-methylpyridin-2-yl]methanol

Concentrated aqueous HCl (12 mL) was added to a solution of tert-butyl{[6-[(tert-butoxycarbonyl)amino]-2-(hydroxymethyl)-4-methylpyridin-3-yl]methyl}-carbamate (0.400 g, 1.09 mmol; see step (a) above) in THF (25 mL) and the reaction mixture was stirred at rt overnight. The mixture was concentrated under reduced pressure and the residue was washed with diethyl ether/EtOH 3:1 to give the hydrochloride salt of the title compound (0.248 g, 95%).

### Preparation 7

### [6-Amino-3-(aminomethyl)-4-methylpyridin-2-yl]methyl acetate

A solution of (6-[(tert-butoxycarbonyl)amino]-3-{[(tert-butoxycarbonyl)amino]-methyl}-4-methylpyridin-2-yl)methyl acetate (0.098 g, 0.24 mmol; see Preparation 5 above) in DCM/TFA (4:1, 2 mL) was stirred for 3 h at rt. The reaction mixture was concentrated under reduced pressure before being redissolved in 4 M HCl in THF. Concentration under reduced pressure gave the hydrochloride salt of the sub-title compound (0.054 g, 80%).

### Preparation 8

### [6-Amino-3-(aminomethyl)-4-methylpyridin-2-yl]methyl benzoate

A solution of benzoyl chloride (0.026 g, 0.19 mmol) in DCM (1 mL) was added dropwise to a solution of triethylamine (0.03 mL, 0.22 mmol) and tert-butyl{[6-[(tert-butoxycarbonyl)amino]-2-(hydroxymethyl)-4-methylpyridin-3-yl]methyl}-carbamate (0.068 g, 0.19 mmol; see Preparation 6(a) above) in DCM (4 mL). The reaction mixture was stirred at rt for 2 days. The resulting solution was washed twice with sulfuric acid (0.5 M) and then with saturated aqueous Na₂CO₃. The organic layer was dried over Na₂SO₄, filtered and evaporated. The crude product thereby obtained was dissolved in THF (5 mL). Concentrated aqueous HCl (2 mL) was added to the resulting solution and the reaction mixture was stirred at rt overnight before being concentrated under reduced pressure to provide a residue that was washed with diethyl ether/EtOH (3:1). This yielded the hydrochloride salt of the title compound (0.050 g, 79%).

### Preparation 9

### Ethyl 2-[1-[(1-ethyl-2-oxo-3-pyridyl)methylamino]-4-methyl-2-oxo-5,6-dihydropyridin-3-yl]acetate

### (a) 1-Ethyl-2-oxo-pyridine-3-carbaldehyde

Iodotrimethylsilane (1.99 mL, 14.0 mmol) was added to a solution of 2-methoxynicotinaldehyde (2.00 g, 14.58 mmol) in dry CHCl₃ (15 mL). The solution was heated at 60° C. for 1 hour and then quenched with dry MeOH (2.5 mL). After concentration, the solid residue was recrystallized with TBME/EtOH. The remaining white solid was dissolved in dry DME (25 mL) and IC₂CO₃ (1.89 g, 13.70 mmol) was added. Ethyl iodide (0.62 mL, 7.70 mmol) was added dropwise while the reaction was heated to reflux. After 8 hours, the reaction mixture was cooled to RT, filtered and evaporated. Purification using flash chromatography (EtOAc) gave 1.154 g (52%) of 1-ethyl-2-oxo-pyridine-3-carbaldehyde.

### (b) Ethyl 2-[1-[(1-ethyl-2-oxo-3-pyridyl)methylamino]-4-methyl-2-oxo-5,6-dihydropyridin-3-yl]acetate

A solution of 1-ethyl-2-oxo-pyridine-3-carbaldehyde (0.211 g, 1.395 mmol) in MeOH (6 mL) was added to a suspension of (1-amino-4-methyl-2-oxo-1,2,5,6-tetrahydropyridin-3-yl)acetic acid ethyl ester (0.355 g, 1.674 mmol) in MeOH (4 mL). AcOH (0.4 mL) was added. The reaction was stirred for 30 minutes and then a solution of sodium cyanoborohydride (0.438 g, 6.975 mmol) in MeOH/AcOH (5 mL/0.6 mL) was added. The reaction mixture was stirred overnight at RT. After evaporation, the residue was dissolved in EtOAc/water. The organic phase was washed with sat. NaHCO₃, water and brine. The combined water phases were extracted with EtOAc and the organic phase was washed with water and brine. The combined organic phases were dried over MgSO₄, filtered and evaporated to give 0.393 g of crude material. Purification using flash chromatography (EtOAc) gave 0.165 g (34%) of the title compound.

### Preparation 10

The following compounds were prepared using procedures analogous to the procedure described in Preparation 9, employing the appropriate aldehyde, either commercially available or from Preparation 12, 13 or 14, in place of 1-ethyl-2-oxo-pyridine-3-carbaldehyde.
(a) Ethyl 2-[1-[(1-ethyl-4-oxo-3-pyridyl)methylamino]-4-methyl-2-oxo-5,6-dihydropyridin-3-yl]acetate
(b) Ethyl 2-[1-[(1-ethyl-5-fluoro-2-oxo-3-pyridyl)methylamino]-4-methyl-2-oxo-5,6-dihydropyridin-3-yl]acetate
(c) Ethyl 2-[4-methyl-1-[(2-morpholino-3-pyridyl)methylamino]-2-oxo-5,6-dihydropyridin-3-yl]acetate
(d) Ethyl 2-[1-[(1-ethyl-3-methyl-pyrazol-4-yl)methylamino]-4-methyl-2-oxo-5,6-dihydropyridin-3-yl]acetate
(e) Ethyl 2-[1-[(5-chloro-1,3-dimethyl-pyrazol-4-yl)methylamino]-4-methyl-2-oxo-5,6-dihydropyridin-3-yl]acetate
(f) Ethyl 2-[1-[[2,2-difluoro-2-(6-methoxy-pyridin-2-yl)ethyl]amino]-4-methyl-2-oxo-5,6-dihydropyridin-3-yl]acetate

### Preparation 11

The following compounds were prepared using procedures analogous to the procedure described in Preparation 3, employing the appropriate ester from Preparation 9, 10 or 15 in place of ethyl{1-[(2,2-difluoro-2-pyridin-2-ylethyl)amino]-4-methyl-2-oxo-1,2,5,6-tetra-hydropyridin-3-yl}acetate.
(a) 2-[1-[(1-Ethyl-2-oxo-3-pyridyl)methylamino]-4-methyl-2-oxo-5,6-dihydropyridin-3-yl]acetic acid
(b) 2-[1-[(1-Ethyl-4-oxo-3-pyridyl)methylamino]-4-methyl-2-oxo-5,6-dihydropyridin-3-yl]acetic acid
(c) 2-[1-[(1-Ethyl-5-fluoro-2-oxo-3-pyridyl)methylamino]-4-methyl-2-oxo-5,6-dihydropyridin-3-yl]acetic acid
(d) 2-[4-Methyl-1-[(2-morpholino-3-pyridyl)methylamino]-2-oxo-5,6-dihydropyridin-3-yl]acetic acid
(e) 2-[1-[(1-Ethyl-3-methyl-pyrazol-4-yl)methylamino]-4-methyl-2-oxo-5,6-dihydropyridin-3-yl]acetic acid
(e) 2-[1-[(5-Chloro-1,3-dimethyl-pyrazol-4-yl)methylamino]-4-methyl-2-oxo-5,6-dihydropyridin-3-yl]acetic acid
(f) 2-[1-[[2,2-Difluoro-2-(6-oxo-1H-pyridin-2-yl)ethyl]amino]-4-methyl-2-oxo-5,6-dihydropyridin-3-yl]acetic acid

### Preparation 12

### 1-Ethyl-5-fluoro-2-oxo-pyridine-3-carbaldehyde

### (a) 5-Fluoro-2-oxo-pyridine-3-carbaldehyde

A flask containing 5-fluoro-2-methoxy-pyridine-3-carbaldehyde (1.551 g, 10.0 mmol) and pyridine hydrochloride (6.9 g, 60.0 mmol) was heated at 145° C. for 10 minutes. The molten mixture was congealed when cooled. Water and EtOAc were added and the pyridine hydrochloride was removed with the water-phase. The water phase was then extracted with EtOAc (3×) and the combined organic phases were dried over MgSO₄. Evaporation gave 0.592 g (42%) of 5-fluoro-2-oxo-pyridine-3-carbaldehyde.

### (b) 1-Ethyl-5-fluoro-2-oxo-pyridine-3-carbaldehyde

K₂CO₃ (0.830 g, 6.00 mmol) was added to a solution of 5-Fluoro-2-oxo-pyridine-3-carbaldehyde (0.424, 3.00 mmol) in dry DME (10 mL). Ethyl iodide (0.303 mL, 3.75 mmol) was added dropwise while the reaction was heated to reflux. After 8 hours the reaction was cooled to RT, filtered and evaporated. Purification using flash chromatography (heptane/EtOAc, 10-100%) gave 0.249 g (49%) of the title compound.

### Preparation 13

The following compounds were prepared using procedures analogous to the procedure described in Preparation 12, employing the appropriate aldehyde in place of 5-fluoro-2-oxo-pyridine-3-carbaldehyde.

### (a) 1-Ethyl-4-oxo-pyridine-3-carbaldehyde

### Preparation 14

### 6-(1,1-Difluoro-2,2-dihydroxy-ethyl)-2-methoxy-pyridin

### (a) Ethyl 2,2-difluoro-2-(6-methoxy-pyridin-2-yl)acetate

Copper bronze (4.19 g, 66.0 mmol) was added to a solution of ethyl bromodifluoroacetate (6.39 g, 31.5 mmol) and 2-bromo-6-methoxy-pyridin (5.64 g, 30.0 mmol) in DMSO (24 mL). The mixture was heated to 50° C. and stirred at this temperature for 2 hours. The reaction mixture was cooled to RT and diluted with isopropyl acetate (45 mL). A solution of potassium dihydrogen phosphate (1.27 M; 69 mL) was added and the mixture stirred for 30 minutes before filtering. The copper salts were washed with isopropyl acetate (45 mL). The filtrate layers were separated and the organic layer washed with water (2×45 mL). The organic layer was evaporated to an orange oil. Purification using flash chromatography (hexane/TBME, 5-30%) gave 3.27 g (47%) of ethyl 2,2-difluoro-2-(6-methoxy-pyridin-2-yl)acetate.

### (b) 6-(1,1-Difluoro-2,2-dihydroxy-ethyl)-2-methoxy-pyridin

NaBH₄ (0.493 g, 13.03 mmol) was added in portions to a solution of ethyl 2,2-difluoro-2-(6-methoxy-pyridin-2-yl)acetate (2.95 g, 12.78 mmol, prepared using a procedure analogous to the procedure described in Preparation 14 (a)) and LiCl (2.71 g, 63.88 mmol) in MeOH (40 mL) at 0° C. After stirring for 30 minutes the cooling bath was removed and stirring continued for 1 hour. The reaction was quenched with 2M HCl (20 mL) and the solution was concentrated. The residue was suspended in a small amount of EtOH and partitionated between 1M HCl and MTBE, the aqueous layer was extracted with MTBE and the combined organic layers were washed with brine and evaporated. Purification using flash chromatography (heptane/acetone, 10-60%) gave 0.663 g (25%) of the title compound.

### Preparation 15

The following compound was prepared using a procedure analogous to the procedure described in Preparation 12 (a), using ethyl 2-[1-[[2,2-difluoro-2-(6-methoxy-pyridin-2-yl)ethyl]amino]-4-methyl-2-oxo-5,6-dihydropyridin-3-yl]acetate in place of 5-fluoro-2-methoxy-pyridine-3-carbaldehyde.

### (a) Ethyl 2-[1-[[2,2-difluoro-2-(6-oxo-1-pyridin-2-yl)ethyl]amino]-4-methyl-2-oxo-5,6-dihydropyridin-3-yl]acetate

### Preparation 16

### tert-Butyl N-[5-(aminomethyl)-6-(hydroxymethyl)-4-methyl-2-pyridyl]carbamate

### (a) tert-Butyl N-[5-[(9H-fluoren-9-ylmethoxycarbonylamino)methyl]-4,6-dimethyl-2-pyridyl]carbamate

9-fluoromethyl-succinimidyl-carbonate (4.33 g, 12.83 mmol) and acetone (80 mL) was added to a solution of tert-butyl N-[5-(aminomethyl)-4,6-dimethyl-2-pyridyl]carbamate (2.93 g, 11.67 mmol) in water (80 mL). Sodium carbonate (1.24 g, 11.67 mmol) was added and the reaction mixture was stirred overnight at RT. The solution was concentrated and then extracted with diethylether. The aqueous phase was acidified using 10% KHSO₄ solution and extracted with EtOAc. The combined organic phases were washed with brine, dried over MgSO₄ and concentrated to give 5.42 g (98%) of tert-butyl N-[5-[(9H-fluoren-9-ylmethoxycarbonylamino)methyl]-4,6-dimethyl-2-pyridyl]carbamate.

### (b) tert-Butyl N-[5- [(9H-fluoren-9-ylmethoxycarbonylamino)methyl]-6-(hydroxymethyl)-4-methyl-2-pyridyl]carbamate

tert-Butyl N-[5-[(9H-fluoren-9-ylmethoxycarbonylamino)methyl]-6-(hydroxymethyl)-4-methyl-2-pyridyl]carbamate was prepared using a procedure analogous to the procedure described in Preparations 4, 5 and 6a using tert-butyl N-[5-[(9H-fluoren-9-ylmethoxycarbonylamino)methyl]-4,6-dimethyl-2-pyridyl]carbamate in place of tert-butyl({6-[(tert-butoxycarbonyl)amino]-2,4-dimethylpyridin-3-yl}methyl)carbamate.

### (c) tert-Butyl N-[5-(aminomethyl)-6-(hydroxymethyl)-4-methyl-2-pyridyl]carbamate

Piperidine (1.075 mL) was added to a solution of tert-butyl N-[5-[(9H-fluoren-9-ylmethoxycarbonylamino)methyl]-6-(hydroxymethyl)-4-methyl-2-pyridyl]carbamate (1.053 g, 2.15 mmol) in DMF (20 mL). The resulting solution was stirred at RT for 1 hour. The solvent was removed by evaporation and the residue was purified by flash chromatography (DCM/MeOH, 10:1+2% Et₃N) to give 0.459 g (80%) of the title compound.

### Synthesis of Compounds of Formula I

### Example 1

### N-{[6-Amino-2-(hydroxymethyl)-4-methylpyridin-3-yl]methyl}-2-{1-[(2,2-difluoro-2-pyridin-2-ylethyl)amino]-4-methyl-2-oxo-1,2,5,6-tetrahydropyridin-3-yl}acetamide

A solution of {1-[(2,2-difluoro-2-pyridin-2-ylethyl)amino]-4-methyl-2-oxo-1,2,5,6-tetrahydropyridin-3-yl}acetic acid (0.037 g, 0.114 mmol; see Preparation 3 above) in DMF (2 mL) was added to [6-amino-3-(aminomethyl)-4-methyl-pyridin-2-yl]methanol (0.041 g, 0.171 mmol; see Preparation 6 above) and HOBT-hydrate (0.026 g, 0.171 mmol). Triethylamine (0.023 mL, 0.171 mmol) was added, followed by EDC (0.033 g, 0.171 mmol), and the reaction mixture was stirred at rt for 2 days. The crude product thereby obtained was purified by preparative HPLC (C8 column, 300×50.8 mm, 50 mL/min, acetonitrile/0.1 M NH₄OAc in water, gradient 20-100% acetonitrile for 20 min) to give 0.020 g (37%) of the title compound.

¹H NMR (400 MHz, CD₃OD): δ 8.66 (d, J=4.6 Hz, 1H), 7.98 (t, J=7.9 Hz, 1H), 7.75 (d, J=7.9 Hz, 1H), 7.55-7.52 (m, 1H), 6.64 (s, 1H), 4.84 (s, 2H), 4.28 (s, 2H), 3.71 (t, J=14.1 Hz, 2H), 3.38 (t, J=7.3 Hz, 2H), 3.25 (s, 2H), 2.42-2.37 (m, 5H), 1.91 (s, 3H).

HRMS (ESI) calculated for C₂₃H₂₉N₆O₃F₂ 475.2269 (M+H)⁺. found 475.228.

### Example 2

### (6-Amino-3-{[({1-[(2,2-difluoro-2-pyridin-2-ylethyl)amino]-4-methyl-2-oxo-1,2,5,6-tetrahydropyridin-3-yl}acetyl)amino]methyl}-4-methylpyridin-2-yl)-methyl acetate

The title compound was prepared using the procedure set out in Example 1, employing [6-amino-3-(aminomethyl)-4-methylpyridin-2-yl]methyl acetate (see Preparation 7 above) in place of [6-amino-3-(aminomethyl)-4-methylpyridin-2-yl]methanol.

¹H NMR (400 MHz, CD₃OD): δ 8.66 (d, J=4.4 Hz, 1H), 7.97 (t, J=1.2, 7.8 Hz, 1H), 7.74 (d, J=7.8 Hz, 1H), 7.55-7.52 (m, 1H), 6.45 (s, 1H), 5.11 (s, 2H), 4.35 (s, 2H), 3.71 (t, J=14.1 Hz, 2H), 3.38 (t, J=7.3 Hz, 2H), 3.26 (s, 2H), 2.39 (t, J=7.3 Hz, 2H), 2.29 (s, 3H), 2.11 (s, 3H), 1.92 (s, 3H).

HRMS (ESI) calculated for C₂₅H₃₁N₆O₄F₂ 517.2375 (M+H)⁺. found 517.2331.

### Example 3

### (6-Amino-3-{[({1-[(2,2-difluoro-2-pyridin-2-ylethyl)amino]-4-methyl-2-oxo-1,2,5,6-tetrahydropyridin-3-yl}acetyl)amino]methyl}-4-methylpyridin-2-yl)-methyl benzoate

The title compound was prepared using the procedure set out in Example 1, and employing [6-amino-3-(aminomethyl)-4-methylpyridin-2-yl]methyl benzoate (see Preparation 8 above) in place of [6-amino-3-(aminomethyl)-4-methylpyridin-2-yl]methanol

¹H NMR (400 MHz, CD₃OD): δ 8.65 (d, J=4.4 Hz, 1H), 8.06 (d, J=7.3 Hz, 2H), 7.96 (t, J=1.4, 7.9 Hz, 1H), 7.73 (d, J=7.9 Hz, 1H), 7.62 (t, J=7.5 Hz, 1H), 7.53-7.47 (m, 3H), 6.49 (s, 1H), 5.36 (s, 2H), 4.43 (s, 2H), 3.70 (t, J=14.1 Hz, 2H), 3.37 (t, J=7.3 Hz, 2H), 3.17 (s, 2H), 2.37 (t, J=7.3 Hz, 2H), 2.31 (s, 3H), 1.86 (s, 3H).

HRMS (ESI) calculated for C₃₀H₃₃N₆O₄F₂ 579.2531 (M+H)⁺. found 579.2569.

HRMS (ESI) calculated for C₃₀H₃₃N₆O₄F₂ 579.2531 (M+H)⁺. found 579.2569.

### Example 4

Using procedures analogous to those set out in Example 1 above, employing an acid reagent from Preparation 11 above, the following compounds were prepared.

### N-[[6-Amino-2-(hydroxymethyl)-4-methyl-3-pyridyl]methyl]-2-[1-[(1-ethyl-4-oxo-3-pyridyl)methylamino]-4-methyl-2-oxo-5,6-dihydropyridin-3-yl]acetamide

¹H NMR (400 MHz, CD₃OD): 7.85 (d, 1H), 7.74-7.71 (dd, 1H), 6.56 (s, 1H), 6.39-6.37 (d, 1H), 4.76 (s, 2H), 4.20 (s, 2H), 4.01-3.95 (q, 2H), 3.78 (s, 2H), 3.48 3.46 (dd, 2H), 3.16 (s, 2H), 2.46-2.43 (dd, 2H), 2.34 (s, 3H), 1.87 (s, 3H), 1.39-1.35 (t, 3H)

### N-[[6-Amino-2-(hydroxymethyl)-4-methyl-3-pyridyl]methyl]-2-[1-[(5-chloro-1,3-dimethyl-pyrazol-4-yl)methylamino]-4-methyl-2-oxo-5,6-dihydropyridin-3-yl]acetamide

¹H NMR (400 MHz, CD₃OD): 6.46 (s, 1H), 4.66 (s, 2H), 4.26 (s, 2H), 3.75 (s, 2H), 3.71 (s, 3H), 3.36-3.32 (t, 2H), 2.36 (t, 2H), 2.29 (s, 2H), 2.19 (s, 2H), 1.91 (s, 3H), 1.88 (s, 3H).

HRMS (ESI) calculated for C₂₂H₃₀N₇O₃ 475.98 (M+H)⁺. found 476.2171.

### N-[[6-Amino-2-(hydroxymethyl)-4-methyl-3-pyridyl]methyl]-2-[4-methyl-1-[(2-morpholino-3-pyridyl)methylamino]-2-oxo-5,6-dihydro-pyridin-3-yl]acetamide

¹H NMR (400 MHz, CD₃OD): 8.17-8.15 (dd, 1H), 7.74-7.72 (d, 1H), 7.03-7.01 (dd, 1H), 6.41 (s, 1H), 4.63 (s, 2H), 4.27 (s, 2H), 3.97 (s, 2H), 3.97-3.80 (m, 4H), 3.43-3.40 (t, 2H), 3.22 (s, 3H), 3.17-3.14 (t, 4H), 2.42-2.38 (t, 2H), 2.27 (s, 3H), 1.92 (s, 3H), 1.88 (s, 3H)

### N-[[6-Amino-2-(hydroxymethyl)-4-methyl-3-pyridyl]methyl]-2-[1-[(1-ethyl-3-methyl-pyrazol-4-yl)methylamino]-4-methyl-2-oxo-5,6-dihydropyridin-3-yl]acetamide

¹H NMR (400 MHz, CD₃OD): 7.46 (s, 1H), 6.40 (s, 1H), 4.62° (s, 2H), 4.29 (s, 2H), 4.06-4.01 (m, 2H), 3.75 (s, 2H), 3.39-3.35 (t, 2H), 3.23 (s, 2H), 2.38-2.35 (t, 2H), 2.27 (s, 3H), 2.20 (s, 3H), 1.91 (s, 3H), 1.38-1.32 (m, 3H)

HRMS (ESI) calculated for C₂₃H₃₃N₇O₃ 455.56 (M+H)⁺. found 456.2739.

### Example 5

### N-[[6-amino-2-(hydroxymethyl)-4-methyl-3-pyridyl]methyl]-2-[1-[(1-ethyl-2-oxo-3-pyridyl)methylamino]-4-methyl-2-oxo-5,6-dihydropyridin-3-yl]acetamide

### (a) tert-butyl N-[5-[[[2-[1-[(1-ethyl-2-oxo-3-pyridyl)methylamino]-4-methyl-2-oxo-5,6-dihydropyridin-3-yl]acetyl]amino]methyl]-6-(hydroxymethyl)-4-methyl-2-pyridyl]carbamate

TEA (0.066 mL, 0.47 mmol) was added to a solution of 2-[1-[(1-Ethyl-2-oxo-3-pyridyl)methylamino]-4-methyl-2-oxo-5,6-dihydropyridin-3-yl]acetic acid (0.050 g, 0.157 mmol) in dry DCM (1 mL) at 0° C. A solution of PyBOP (0.081 g, 0.157 mmol) in dry DCM (1 mL) was added dropwise. After 5 min, a solution of tert-Butyl N-[5-(aminomethyl)-6-(hydroxymethyl)-4-methyl-2-pyridyl]carbamate (0.042 g, 0.157 mmol) in dry DCM (1 mL) was added and the reaction was allowed to reach rt. After stirring overnight, water was added and the phases separated through a phase separator. The organic phase was washed with water, dried through a phase separator and evaporated to give 0.071 g (80%) of tert-butyl N-[5-[[[2-[1-[(1-ethyl-2-oxo-3-pyridyl)methylamino]-4-methyl-2-oxo-5,6-dihydropyridin-3-yl]acetyl]amino]methyl]-6-(hydroxymethyl)-4-methyl-2-pyridyl]carbamate. The crude product was used without further purification in the next step.

### (b) N-[[6-amino-2-(hydroxymethyl)-4-methyl-3-pyridyl]methyl]-2-[1-[(1-ethyl-2-oxo-3-pyridyl)methylamino]-4-methyl-2-oxo-5,6-dihydropyridin-3-yl]acetamide

Concentrated aqueous HCl (2 mL) was added to a solution of tert-butyl N-[5-[[[2-[1-[(1-ethyl-2-oxo-3-pyridyl)methylamino]-4-methyl-2-oxo-5,6-dihydropyridin-3-yl]acetyl]amino]methyl]-6-(hydroxymethyl)-4-methyl-2-pyridyl]carbamate (0.085 g, 0.150 mmol) in THF (4 mL), and the reaction mixture was stirred at RT overnight. After evaporation the residue was washed with ether/EtOH (3:1) and the crude product was collected as the hydrogen chloride salt. The crude product was purified by preparative HPLC (C8 column, 300×50.8 mm, 20 mL/min, MeCN/0.1 M NHOAc in water, gradient 5-60% MeCN for 25 min) to give 0.014 g (19%) of the title compound.

¹H NMR (400 MHz, D₂O): d 7.61 (d, J=6.9 Hz, 1H), 7.50 (d, J=6.9 Hz, 1H), 6.66 (s, 1H), 6.42 (t, J=6.9 Hz, 1H), 4.74 (s, 2H), 4.28 (s, 2H), 4.04 (q, J=7.3 Hz, 2H), 3.86 (s, 2H), 3.50 (t, J=7.3 Hz, 2H), 3.21 (s, 2H), 2.49 (t, J=7.3 Hz, 2H), 2.31 (s, 3H), 1.89 (s, 3H), 1.31 (t, J=7.3 Hz, 3H).

HRMS (ESI) calculated for C₂₄H₃₂N₆O₄ 469.2563 (M+H)⁺. found 469.2556.

### N-[[6-amino-2-(hydroxymethyl)-4-methyl-3-pyridyl]methyl]-2-[1-[(1-ethyl-5-fluoro-2-oxo-3-pyridyl)methylamino]-4-methyl-2-oxo-5,6-dihydropyridin-3-yl]acetamide

The title compound was prepared using the procedure set out in example 5, and employing 2-[1-[(1-Ethyl-5-fluoro-2-oxo-3-pyridyl)methylamino]-4-methyl-2-oxo-5,6-dihydropyridin-3-yl]acetic acid in place of 2-[1-[(1-Ethyl-2-oxo-3-pyridyl)methylamino]-4-methyl-2-oxo-5,6-dihydropyridin-3-yl]acetic acid

¹H NMR (400 MHz, D₂O): d 7.68 (s, 1H), 7.56 (d, 1H), 6.76 (s, 1H), 4.85 (s, 2H), 4.25 (s, 2H), 4.03 (q, J=7.3 Hz, 2H), 3.88 (s, 2H), 3.53 (t, J=7.1 Hz, 2H), 3.22 (s, 2H), 2.53 (t, J=7.1 Hz, 2H), 2.37 (s, 3H), 1.90 (s, 3H), 1.32 (t, J=7.3 Hz, 3H).

HRMS (ESI) calculated for C₂₄H₃₂N₆O₄F 487.2469 (M+H)⁺. found 487.2481.

### N-[[6-Amino-2-(hydroxymethyl)-4-methyl-3-pyridyl]methyl]-2-[1-[[2,2-difluoro-2-(6-oxo-1H-pyridin-2-yl)ethyl]amino]-4-methyl-2-oxo-5,6-dihydropyridin-3-yl]acetamide

The title compound was prepared using the procedure set out in example 5, and employing 2-[1-[[2,2-Difluoro-2-(6-oxo-1H-pyridin-2-yl)ethyl]amino]-4-methyl-2-oxo-5,6-dihydropyridin-3-yl]acetic acid in place of 2-[1-[(1-Ethyl-2-oxo-3-pyridyl)methylamino]-4-methyl-2-oxo-5,6-dihydropyridin-3-yl]acetic acid

¹H NMR (400 MHz, MeOD): δ 7.68 (t, J=8.9 Hz, 1H), 6.78 (d, J=8.9 Hz, 1M), 6.70 (d, J=8.9 Hz, 1H), 6.43 (s, 1H), 4.68, (s, 2H), 4.33 (s, 2H), 3.57 (t, J=13.3 Hz, 2H), 3.48 (t, J=7.3 Hz, 2H), 3.28 (s, 2H), 2.47 (t, J=7.3 Hz, 2H), 2.30 (s, 3H), 1.96 (s, 3H)

HRMS (ESI) calculated for C₂₃H₂₉N₆O₄F₂ 491.2218 (M+H)⁺. found 491.2227.

### Example 6

Compounds of the Examples were tested in Test B above and were found to exhibit IC₅₀TT values of less than 50 μM. Indeed, the compound of Example 1 was found to exhibit an IC₅₀ value of 4.7 DM.

### Example 7

The title compounds of Example 2 and 3 were tested in Test F above and were found to be converted to the corresponding active inhibitor (title compound of Example 1) in liver microsomes from humans and from rats.

### Abbreviations

AcOH=acetic acid
aq.=aqueous
AUC=area under the curve
Boc=tert-butyloxycarbonyl
BSA=bovine serum albumin
d=(in relation to NMR) doublet
DCC=dicyclohexyl carbodiimide
DCE=1,2-dichloroethane
DCM=dichloromethane
DIPEA=diisopropylethylamine
DMAP=4-(N,N-dimethyl amino)pyridine
DME=1,2-dimethoxyethane
DMF=dimethylformamide
DMSO=dimethylsulfoxide
DVT=deep vein thrombosis
EDC=1-(3-dimethylaminopropyl)-3-ethylcarbodiimide hydrochloride
ESI=electron spray ionisation
Et=ethyl
ether=diethyl ether
Et₃N=triethylamine
EtOAc=ethyl acetate
EtOH=ethanol
Et₂O=diethyl ether
h=hour(s)
HATU=O-(azabenzotriazol 1-yl)-N,N,N′,N′-tetramethyluronium hexafluorophosphate
HBTU=[N,N,N′,N′-tetramethyl-O-(benzotriazo-1-yl)uronium hexafluorophosphate]
HCl=hydrochloric acid, hydrogen chloride gas or hydrochloride salt (depending on context)
HOAt=1-hydroxy-7-azabenzotriazole
HOBt=1-hydroxybenzotriazole
HPLC=high performance liquid chromatography
HRMS=high resolution mass spectrometry
LC=liquid chromatography
mCPBA=meta-chloroperbenzoic acid
Me=methyl
MeCN=acetonitrile
MeOH=methanol
min=minute(s)
MS=mass spectroscopy
NADH=nicotinamide adenine dinucleotide, reduced form
NADPH=nicotinamide adenine dinucleotide phosphate, reduced form
NBS=N-Bromosuccinimide
NIH=National Institute of Health (US)
NIHU=National Institute of Health units
OAc=acetate
PCC=pyridinium chlorochromate
Ph=phenyl
Pr=propyl
PyBOP=(benzotriazol-1-yloxy)tripyrrolidinophosphonium hexafluorophosphate
rt/RT=room temperature
SOPs=standard operating procedures
TBME=tert-butyl methyl ether
TBTU=[N,N,N′,N′-tetramethyl-O-(benzotriazol 1-yl)uronium tetrafluoroborate]
TEA=triethylamine
TFA=trifluoroacetic acid
THF tetrahydrofuran

Prefixes n, s, i and t have their usual meanings: normal, secondary, iso and tertiary. The prefix c means cyclo.

1. A compound of formula I 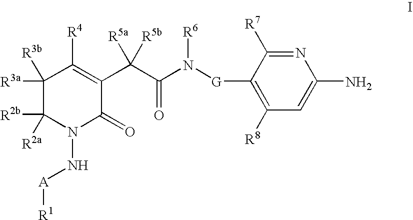
wherein
A represents C(O), S(O)₂, C(O)O (in which latter group the 0 moiety is attached to R¹), C(O)NH, S(O)₂NH (in which latter two groups the NH moiety is attached to R¹), a direct bond or C₁₋₆ alkylene (which latter group is optionally substituted, at the C-atom to which the NH moiety is attached, by C(O)OR^{A} or C(O)N(H)R^{A});
R^{A} represents H or C₁₋₄ alkyl;
R¹ represents
(a) C₁₋₁₀ alkyl, C₂₋₁₀ alkenyl, C₂₋₁₀ alkynyl (which latter three groups are optionally substituted by one or more substituents selected from halo, CN, C₃₋₁₀ cycloalkyl (optionally substituted by one or more substituents selected from halo, OH, ═O, C₁₋₆ alkyl, C₁₋₆ alkoxy and aryl), OR^{9a}, S(O)ₙR^{9b}, S(O)₂N(R^{9c})(R^{9d}), N(R^{9e})S(O)₂R^{9f}, N(R^{9g})(R^{9h}), B¹—C(O)—B²—R⁹ⁱ, aryl and Het¹),
(b) C₃₋₁₀ cycloalkyl or C₄₋₁₀ cycloalkenyl, which latter two groups are optionally substituted by one or more substituents selected from halo, ═O, CN, C₁₋₁₀ alkyl, C₃₋₁₀ cycloalkyl (optionally substituted by one or more substituents selected from halo, OH, ═O, C₁₋₆ alkyl, C₁₋₆ alkoxy and aryl), OR^{9a}, S(O)ₙR^{9b}, S(O)₂N(R^{9c})(R^{9d}), N(R^{9e})S(O)₂R^{9f}, N(R^{9g})(R^{9h}), B³—C(O)—B⁴—R⁹ⁱ, aryl and Het²,
(c) aryl, or
(d) Het³;
R^{9a} to R⁹ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, aryl and Het⁴),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, aryl and Het⁵),
(d) aryl or
(e) Het⁶,
provided that R^{9b} does not represent H when n is 1 or 2;
R^{2a}, R^{2b}, R^{3a} and independently represent H, F, C₁₋₃ alkyl or (CH₂)₀₋₃O(C₁₋₃ alkyl) (which latter two groups are optionally substituted by one OH group or one or more F atoms), or one of R^{2a} and R^{2b}, together with one of R^{3a} and R^{3b}, represents C₁₋₄ n-alkylene;
R⁴ represents C₁₋₄ alkyl optionally substituted by one or more halo substituents;
R^{5a} and R^{5b} independently represent H, F or methyl (which latter group is optionally substituted by one or more F atoms);
R⁶ represents H or C₁₋₄ alkyl (which latter group is optionally substituted by one or more substituents selected from halo and OH),
G represents C₁₋₄ alkylene;
R⁷ and R⁸ independently represent C₁₋₄ alkyl optionally substituted by OR¹⁰, provided that at least one of R⁷ and R⁸ is substituted by OR¹⁰;
R¹⁰ represents H, —C(O)—X—R¹¹ or C₁₋₆ alkyl (which latter group is optionally substituted by one or more substituents selected from halo and C₁₋₃ alkoxy);
X represents a direct bond, O, S or NH;
R¹¹ represents
(a) C₁₋₁₀ alkyl, C₂₋₁₀ alkenyl, C₂₋₁₀ alkynyl (which latter three groups are optionally substituted by one or more substituents selected from halo, CN, C₃₋₁₀ cycloalkyl, C₄₋₁₀ cycloalkenyl (which latter two groups are optionally substituted by one or more substituents selected from halo and C₁₋₄ alkyl), OR^{12a}, C(O)OR^{12b}, C(O)N(R^{12c})(R^{12d}), aryl and Het⁷),
(b) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, aryl and Het⁸),
(c) aryl or
(d) Het⁹;
R^{12a} to R^{12d} independently represent H or C₁₋₆ alkyl;
each aryl independently represents a C₆₋₁₀ carbocyclic aromatic group, which group may comprise either one or two rings and may be substituted by one or more substituents selected from
(a) halo,
(b) CN,
(c) C₁₋₁₀ alkyl, C₂₋₁₀ alkenyl, C₂₋₁₀ alkynyl (which latter three groups are optionally substituted by one or more substituents selected from halo, OH, C₃₋₁₀ cycloalkyl, C₄₋₁₀ cycloalkenyl (which latter two groups are optionally substituted by one or more substituents selected from halo and C₁₋₄ alkyl), C₁₋₆ alkoxy, C(O)OH, C(O)O—C₁₋₆ alkyl, C(O)NH₂, phenyl (which latter group is optionally substituted by halo) and Het¹⁰),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het¹¹),
(e) OR^{13a},
(f) S(O)ₚR^{13b},
(g) S(O)₂N(R^{13c})(R^{13d}),
(h) N(R^{13e})S(O)₂R^{13f},
(i) N(R^{13g})(R^{13h}),
(j) B⁵—C(O)—B⁶—R¹³ⁱ
(k) phenyl (which latter group is optionally substituted by halo),
(l) Het¹² and
(m) Si(R^{14a})(R^{14b})(R^{14c});
R^{13a} to R¹³ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₃₋₁₀ cycloalkyl, C₄₋₁₀ cycloalkenyl (which latter two groups are optionally substituted by one or more substituents selected from halo and C₁₋₄ alkyl), C₁₋₆ alkoxy, phenyl (which latter group is optionally substituted by halo) and Het¹³),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het¹⁴),
(d) phenyl (which latter group is optionally substituted by halo) or
(e) Het¹⁵,
provided that R^{13b} does not represent H when p is 1 or 2;
Het¹ to Het¹⁵ independently represent 4- to 14-membered heterocyclic groups containing one or more heteroatoms selected from oxygen, nitrogen and/or sulfur, which heterocyclic groups may comprise one, two or three rings and may be substituted by one or more substituents selected from
(a) halo,
(b) CN,
(c) C₁₋₁₀ alkyl, C₂₋₁₀ alkenyl, C₂₋₁₀ alkynyl (which latter three groups are optionally substituted by one or more substituents selected from halo, OH, C₃₋₁₀ cycloalkyl, C₄₋₁₀ cycloalkenyl (which latter two groups are optionally substituted by one or more substituents selected from halo and C₁₋₄ alkyl), C₁₋₆ alkoxy, C(O)OH, C(O)O—C₁₋₆ alkyl, C(O)NH₂, phenyl (which latter group is optionally substituted by halo) and Het^{a}),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{b}),
(e) ═O,
(f) OR^{15a},
(g) S(O)_{q}R^{15b},
(h) S(O)₂N(R^{15c})(R^{15d}),
(i) N(R^{15e})S(O)₂R⁵,
(j) N(R^{15g})(R^{15h}),
(k) B⁷—C(O)—B⁸—R¹⁵ⁱ,
(l) phenyl (which latter group is optionally substituted by halo),
(m) Het^{c} and
(n) Si(R^{16a})(R^{16b})(R^{16c});
R^{15a} to R¹⁵ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₃₋₁₀ cycloalkyl, C₄₋₁₀ cycloalkenyl (which latter two groups are optionally substituted by one or more substituents selected from halo and C₁₋₄ alkyl), C₁₋₆ alkoxy, phenyl (which latter group is optionally substituted by halo) and Het^{d}),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{e}),
(d) phenyl (which latter group is optionally substituted by halo) or
(e) Het^{e},
provided that R^{15b} does not represent H when q is 1 or 2;
Het^{a} to Het^{f} independently represent 5- or 6-membered heterocyclic groups containing one to four heteroatoms selected from oxygen, nitrogen and/or sulfur, which heterocyclic groups may be substituted by one or more substituents selected from halo, ═O and C₁₋₆ alkyl;
B¹ to B⁸ independently represent a direct bond, O, S, NH or N—C₁₋₄ alkyl;
n, p and q independently represent 0, 1 or 2;
R^{14a}, R^{14b}, R^{14c}, R^{16a}, R^{16b} and R^{16c} independently represent C₁₋₆ alkyl or phenyl (which latter group is optionally substituted by halo or C₁₋₄ alkyl);
unless otherwise specified
(i) alkyl, alkenyl, alkynyl, cycloalkyl, cycloalkenyl, alkylene and alkenylene groups, as well as the alkyl part of alkoxy groups, may be substituted by one or more halo atoms, and
(ii) cycloalkyl and cycloalkenyl groups may comprise one or two rings and may additionally be ring-fused to one or two phenyl groups;
or a pharmaceutically-acceptable derivative thereof.

2. A compound as claimed in claim 1 which is a compound of formula Ia 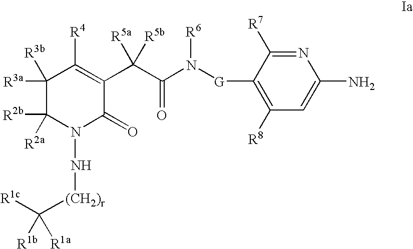
wherein:
R^{1a} represents aryl or Het³;
R^{1b} and R^{1c} independently represent H, halo or methyl;
r represents 0 or 1; and
R^{2a}, R^{2b}, R^{3a}, R^{3b}, R⁴, R^{5a}, R^{5b}, R⁶ to R⁸, G, aryl and Het³ are as defined in claim 1.

3. A compound of formula Ia as claimed in claim 2 wherein:
R^{1a} represents phenyl (optionally substituted by one or more substituents selected from halo, C₁₋₃ alkyl and C₁₋₃ alkoxy (which alkyl and alkoxy groups are optionally substituted by one or more F atoms)) or Het³;
Het³ represents a 5- or 6-membered heterocycle containing, as heteroatom(s), one oxygen or sulfur atom and/or one or two nitrogen atoms, which heterocyclic group may be substituted by one or more substituents selected from halo, C₁₋₃ alkyl and C₁₋₃ alkoxy, which alkyl and alkoxy groups are optionally substituted by one or more F atoms;
R^{1b} and R^{1c} both represent F;
R^{2a}, R^{2b}, R^{3a} and R^{3b} all represent H;
R⁴ represents methyl;
R^{5a} and R^{5b} both represent H;
R⁶ represents H;
G represents C₁₋₂ n-alkylene;
r represents 1;
R⁷ represents CH₂OR¹⁰;
R⁸ represents methyl;
R¹⁰ represents H or —C(O)R¹¹; and
R¹¹ represents C₁₋₂ alkyl (optionally substituted by one or more Cl or F atoms) or phenyl (which latter group is optionally substituted by one or more substituents selected from Cl, F and methyl).

4. A pharmaceutical formulation comprising a compound according to claim 1, or a pharmaceutically acceptable derivative thereof, in admixture with a pharmaceutically acceptable adjuvant, diluent or carrier.

5-6. (canceled)

7. A method of treatment of a hypercoagulability and/or thrombo-embolic disease or condition, which method comprises administration of a therapeutically effective amount of a compound according to claim 1, or a pharmaceutically acceptable derivative thereof, to a person suffering from, or susceptible to, such a disease or condition.

8. A process for the preparation of a compound of formula I as defined in claim 1, which comprises:
(a) coupling of a compound of formula II, 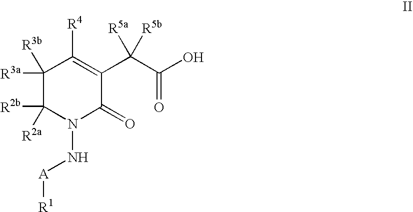
wherein R¹, R^{2a}, R^{2b}, R^{3a}, R^{3b}, R⁴, R^{5a}, R^{5b} and A are as defined in claim 1, with a compound of formula III, 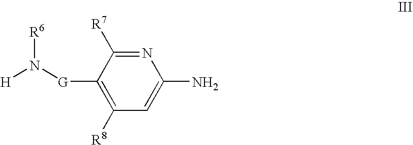
or a derivative thereof that is protected at the 2-amino substituent of the pyridine ring, wherein R⁶ to R⁸ and G are as defined in claim 1;
(b) reaction of a compound of formula IV, 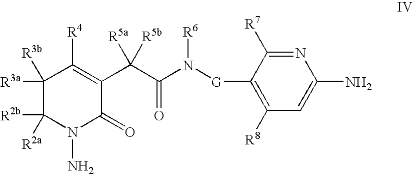
or a derivative thereof that is protected at the 2-amino substituent of the pyridine ring, wherein R^{2a}, R^{2b}, R^{3a}, R^{3b}, R⁴, R^{5a}, R^{5b}, R⁶ to R⁸ and G are as defined in claim 1, with a compound of formula V,
R¹-A-Lg¹ V
wherein Lg¹ represents a leaving group and R¹ and A are as defined in claim 1;
(c) for compounds of formula I in which A represents C(O)NH, reaction of a compound of formula IV, as defined above, or a derivative thereof that is protected at the 2-amino substituent of the pyridine ring, with a compound of formula VI,
R¹—N═C═O VI
wherein R¹ is as defined in claim 1;
(d) for compounds of formula I in which A represents C₁₋₆ alkylene, reaction of a compound of formula IV, as defined above, or a derivative thereof that is protected at the 2-amino substituent of the pyridine ring, with a compound of formula VII,
R¹—C₀₋₅ alkylene-CHO VII
wherein R¹ is as defined in claim 1;
(e) for compounds of formula I in which R⁷ and/or R⁵ represents C₁₋₄ alkyl substituted by —O—C(O)—X—R¹¹, reaction of a corresponding compound of formula I in which R⁷ and/or R³ represents C₁₋₄ alkyl substituted by —OH with a compound of formula VIII,
R¹¹—X—C(O)-Lg² VIII
wherein Lg² represents a leaving group and R¹¹ and X are as defined in claim 1; or
(f) deprotection of a protected derivative of a compound as claimed in claim 1.

9. A compound of formula III, as defined in claim 8, 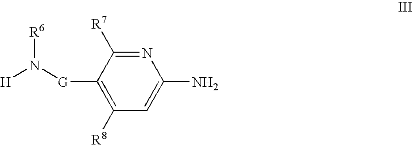
wherein
R⁶ represents H or C₁₋₄ alkyl (which latter group is optionally substituted by one or more substituents selected from halo and OH),
G represents C₁₋₄ alkylene;
R⁷ and R⁸ independently represent C₁₋₄ alkyl optionally substituted by OR¹⁰ provided that at least one of R⁷ and R⁸ is substituted by OR¹⁰;
R¹⁰ represents H, —C(O)—X—R¹¹ or C₁₋₆ alkyl (which latter group is optionally substituted by one or more substituents selected from halo and C₁₋₃ alkoxy);
X represents a direct bond, O, S or NH;
R¹¹ represents
(a) C₁₋₁₀ alkyl, C₂₋₁₀ alkenyl, C₂₋₁₀ alkynyl (which latter three groups are optionally substituted by one or more substituents selected from halo, CN, C₃₋₁₀ cycloalkyl, C₄₋₁₀ cycloalkenyl (which latter two groups are optionally substituted by one or more substituents selected from halo and C₁₋₄ alkyl)OR^{12a}, C(O)OR^{12b}, C(O)N(R^{12c})(R^{12d}) aryl and Het⁷),
(b) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, aryl and Het⁸),
(c) aryl or
(d) Het⁹;
R^{12a} to R^{12d} independently represent H or C₁₋₆ alkyl
each aryl independently represents a C₆₋₁₀ carbocyclic aromatic group which group may comprise either one or two rings and may be substituted by one or more substituents selected from
(a) halo,
(b) CN,
(c) C₁₋₁₀ alkyl, C₂₋₁₀ alkenyl, C₂₋₁₀ alkynyl (which latter three groups are optionally substituted by one or more substituents selected from halo, OH, C₃₋₁₀ cycloalkyl, C₄₋₁₀ cycloalkenyl (which latter two groups are optionally substituted by one or more substituents selected from halo and C₁₋₄ alkyl), C₁₋₆ alkoxy, C(O)OH, C(O)O—C₁₋₆ alkyl, C(O)NH₂ phenyl (which latter group is optionally substituted by halo) and Het¹⁰),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het¹¹),
(e) OR^{13a},
(f) S(O)ₚR^{13b},
(g) S(O)₂N(R^{13c})(R^{13d}),
(h) N(R^{13e})S(O)₂R^{13f},
(i) N(R^{13g})(R^{13h}),
(j) B⁵—C(O)—B⁶—R¹³ⁱ,
(k) phenyl (which latter group is optionally substituted by halo),
(l) Het¹² and
(m) Si(R^{14a})(R^{14b})(R^{14c});
R^{13a} to R¹³ⁱ independently represent at each occurrence
(a) H,
(b) C₁₋₁₀ alkyl, C₂₋₁₀ alkenyl, C₂₋₁₀ alkynyl (which latter three groups are optionally substituted by one or more substituents selected from halo, OH, C₃₋₁₀ cycloalkyl, C₄₋₁₀ cycloalkenyl (which latter two groups are optionally substituted by one or more substituents selected from halo and C₁₋₄ alkyl), C₁₋₆ alkoxy, phenyl (which latter group is optionally substituted by halo) and Het¹³),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het¹⁴),
(d) phenyl (which latter group is optionally substituted by halo) or
(e) Het¹⁵,
provided that R^{13b} does not represent H when p is 1 or 2;
Het⁷ to Het¹⁵ independently represent 4- to 14-membered heterocyclic groups containing one or more heteroatoms selected from oxygen, nitrogen and/or sulfur, which heterocyclic groups may comprise one, two or three rings and may be substituted by one or more substituents selected from
(a) halo,
(b) CN,
(c) C₁₋₁₀ alkyl, C₂₋₁₀ alkenyl, C₂₋₁₀ alkynyl (which latter three groups are optionally substituted by one or more substituents selected from halo, OH, C₃₋₁₀ cycloalkyl, C₄₋₁₀ alkenyl (which latter two groups are optionally substituted by one or more substituents selected from halo and C₁₋₄ alkyl), C₁₋₆ alkoxy, C(O)OH, C(O)O—C₁₋₆ alkyl, C(O)NH₂, phenyl (which latter group is optionally substituted by halo) and Het^{a}),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{b}),
(e) ═O,
(f) OR^{15a},
(g) S(O)_{q}R^{15b},
(h) S(O)₂N(R^{15c})(R^{15d})
(i) N(R^{15e})S(O)₂R^{15f},
(j) N(R^{15g})(R^{15h}),
(k) B⁷—C(O)—B⁸—R¹⁵ⁱ,
(l) phenyl (which latter group is optionally substituted by halo),
(m) Het^{e} and
(n) Si(R^{16a})(R^{16b})(R^{16c});
R^{15a} to R¹⁵ⁱ independently represent at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₃₋₁₀ cycloalkyl, C₄₋₁₀ cycloalkenyl (which latter two groups are optionally substituted by one or more substituents selected from halo and C₁₋₄ alkyl), C₁₋₆ alkoxy, phenyl (which latter group is optionally substituted by halo) and Het^{d}),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{e}),
(d) phenyl (which latter group is optionally substituted by halo) or
(e) Het^{f},
provided that R^{15b} does not represent H when q is 1 or 2;
Het^{a} to Het^{f} independently represent 5- or 6-membered heterocyclic groups containing one to four heteroatoms selected from oxygen, nitrogen and/or sulfur, which heterocyclic groups may be substituted by one or more substituents selected from halo, ═O and C₁₋₆ alkyl
B⁵ to B⁸ independently represent a direct bond, O, S, NH or N—C₁₋₄ alkyl
p and q independently represent 0, 1 or 2;
R^{14a}, R^{14b}, R^{14c}, R^{16a}, R^{16b} and R^{16c} independently represent C₁₋₆ alkyl or phenyl (which latter group is optionally substituted by halo or C₁₋₄ alkyl);
unless otherwise specified
(i) alkyl alkenyl alkynyl cycloalkyl cycloalkenyl alkylene and alkenylene groups, as well as the alkyl part of alkoxy groups, may be substituted by one or more halo atoms and
(ii) cycloalkyl and cycloalkenyl groups may comprise one or two rings and may additionally be ring-fused to one or two phenyl groups;
or a protected derivative thereof.

10. A compound of formula IV, 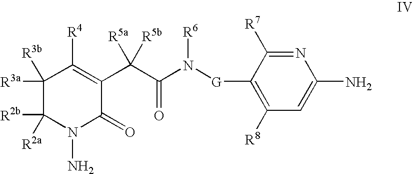
wherein
R^{2a}, R^{2b}, R^{3a} and R^{3b} independently represent H, F, C₁₋₃ alkyl or (CH₂)₀₋₃O(C₁₋₃ alkyl) (which latter two groups are optionally substituted by one OH group or one or more F atoms) or one of R^{2a} and R^{2b} together with one of R^{3a} and R^{3b} represents C₁₋₄ n-alkylene
R⁴ represents C₁₋₄ alkyl optionally substituted by one or more halo substituents;
R^{5a} and R^{5b} independently represent H, F or methyl (which latter group is optionally substituted by one or more F atoms),
R⁶ represents H or C₁₋₄ alkyl (which latter group is optionally substituted by one or more substituents selected from halo and OH),
G represents C₁₋₄ alkylene;
R⁷ and R⁸ independently represent C₁₋₄ alkyl optionally substituted by OR¹⁰ provided that at least one of R⁷ and R⁸ is substituted by OR¹⁰;
R¹⁰ represents H, —C(O)—X—R¹¹ or C₁₋₆ alkyl (which latter group is optionally substituted by one or more substituents selected from halo and C₁₋₃ alkoxy);
X represents a direct bond, O, S or NH;
R¹¹ represents
(a) C₁₋₁₀ alkyl, C₂₋₁₀ alkenyl, C₂₋₁₀ alkynyl (which latter three groups are optionally substituted by one or more substituents selected from halo, CN, C₃₋₁₀ cycloalkyl, C₄₋₁₀ cycloalkenyl (which latter two groups are optionally substituted by one or more substituents selected from halo and C₁₋₄ alkyl), OR^{12a}, C(O)OR^{12b}, C(O)N(R^{12c})(R^{12d}) aryl and Het⁷),
(b) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, aryl and Het⁸),
(c) aryl or
(d) Het⁹;
R^{12a} to R^{12d} independently represent H or C₁₋₆ alkyl each aryl independently represents a C₆₋₁₀ carbocyclic aromatic group which group may comprise either one or two rings and may be substituted by one or more substituents selected from
(a) halo,
(b) CN,
(c) C₁₋₁₀ alkyl, C₂₋₁₀ alkenyl, C₂₋₁₀ alkynyl (which latter three groups are optionally substituted by one or more substituents selected from halo, OH, C₃₋₁₀ cycloalkyl, C₄₋₁₀ cycloalkenyl (which latter two groups are optionally substituted by one or more substituents selected from halo and C₁₋₄ alkyl), C₁₋₆ alkoxy, C(O)OH, C(O)O—C₁₋₆ alkyl, C(O)NH₂ phenyl (which latter group is optionally substituted by halo) and Het¹⁰),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het¹¹),
(e) OR^{13a},
(f) S(O)ₚR^{13b},
(g) S(O)₂N(R^{13c})(R^{13d}),
(h) N(R^{13e})S(O)₂R^{13f},
(i) N(R^{13g})(R^{13h}),
(j) B⁵—C(O)—B⁶—R¹³ⁱ,
(k) phenyl (which latter group is optionally substituted by halo),
(l) Het¹² and
(m) Si(R^{14a})(R^{14b})(R^{14c});
R^{13a} to R¹³ⁱ independently represent at each occurrence
(a) H,
(b) C₁₋₁₀ alkyl, C₂₋₁₀ alkenyl, C₂₋₁₀ alkynyl (which latter three groups are optionally substituted by one or more substituents selected from halo, OH, C₃₋₁₀ cycloalkyl, C₄₋₁₀ alkenyl (which latter two groups are optionally substituted by one or more substituents selected from halo and C₁₋₄ alkyl), C₁₋₆ alkoxy, phenyl (which latter group is optionally substituted by halo) and Het¹³),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het¹⁴),
(d) phenyl (which latter group is optionally substituted by halo) or
(e) Het¹⁵,
provided that R^{13b} does not represent H when p is 1 or 2;
Het⁷ to Het¹⁵ independently represent 4- to 14-membered heterocyclic groups containing one or more heteroatoms selected from oxygen, nitrogen and/or sulfur, which heterocyclic groups may comprise one, two or three rings and may be substituted by one or more substituents selected from
(a) halo,
(b) CN,
(c) C₁₋₁₀ alkyl, C₂₋₁₀ alkenyl, C₂₋₁₀ alkynyl (which latter three groups are optionally substituted by one or more substituents selected from halo, OH, C₃₋₁₀ cycloalkyl, C₄₋₁₀ alkenyl (which latter two groups are optionally substituted by one or more substituents selected from halo and C₁₋₄ alkyl), C₁₋₆ alkoxy, C(O)OH, C(O)O—C₁₋₆ alkyl, C(O)NH₂, phenyl (which latter group is optionally substituted by halo) and Het^{a}),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{b}),
(e) ═O,
(f) OR^{15a},
(g) S(O)_{q}R^{15b},
(h) S(O)₂N(R^{15c})(R^{15d}),
(i) N(R^{15e})S(O)₂R^{15f},
(j) N(R^{15g})(R^{15h}),
(k) B⁷—C(O)—B⁸—R¹⁵ⁱ,
(l) phenyl (which latter group is optionally substituted by halo),
(m) Het^{c} and
(n) Si(R^{16a})(R^{16b})(R^{16c});
R^{15a} to R¹⁵ⁱ independently represent at each occurrence
(a) H,
(b) C₁₋₁₀ alkyl, C₂₋₁₀ alkenyl, C₂₋₁₀ alkynyl (which latter three groups are optionally substituted by one or more substituents selected from halo, OH, C₃₋₁₀ cycloalkyl, C₄₋₁₀ cycloalkenyl (which latter two groups are optionally substituted by one or more substituents selected from halo and C₁₋₄ alkyl), C₁₋₆ alkoxy, phenyl (which latter group is optionally substituted by halo) and Het^{d}),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{e}),
(d) phenyl (which latter group is optionally substituted by halo) or
(e) Het^{e}
provided that R^{15b} does not represent H when q is 1 or 2;
Het^{a} to Het^{f} independently represent 5- or 6-membered heterocyclic groups containing one to four heteroatoms selected from oxygen nitrogen and/or sulfur, which heterocyclic groups may be substituted by one or more substituents selected from halo, ═O and C₁₋₆ alkyl;
B⁵ to B⁸ independently represent a direct bond, O, S, NH or N—C₁₋₄ alkyl
p and q independently represent 0, 1 or 2;
R^{14a}, R^{14b}, R^{14c}, R^{16a}, R^{16b} and R^{16c} independently represent C₁₋₆ alkyl or phenyl (which latter group is optionally substituted by halo or C₁₋₄ alkyl
unless otherwise specified
(i) alkyl alkenyl alkynyl cycloalkyl cycloalkenyl alkylene and alkenylene groups as well as the alkyl part of alkoxy groups may be substituted by one or more halo atoms and
(ii) cycloalkyl and cycloalkenyl groups may comprise one or two rings and may additionally be ring-fused to one or two phenyl groups;
or a protected derivative thereof.

11. A pharmaceutical formulation comprising a compound according to claim 2, or a pharmaceutically acceptable derivative thereof, in admixture with a pharmaceutically acceptable adjuvant, diluent or carrier.

12. A pharmaceutical formulation comprising a compound according to claim 3, or a pharmaceutically acceptable derivative thereof, in admixture with a pharmaceutically acceptable adjuvant, diluent or carrier.

13. A method of treatment of a hypercoagulability and/or thrombo-embolic disease or condition, which method comprises administration of a therapeutically effective amount of a compound according to claim 2, or a pharmaceutically acceptable derivative thereof, to a person suffering from, or susceptible to, such a disease or condition.

14. A method of treatment of a hypercoagulability and/or thrombo-embolic disease or condition, which method comprises administration of a therapeutically effective amount of a compound according to claim 3, or a pharmaceutically acceptable derivative thereof, to a person suffering from, or susceptible to, such a disease or condition.
